(19) United States
(12) Patent Application Publication
(10) Pub. No.: US 2023/0013636 A1
(43) Pub. Date: Jan. 19, 2023

(54) EXOSOMES-BASED THERAPY FOR LIVER FIBROSIS AND OTHER DISEASES WITH FIBROSIS

(71) Applicant: BOARD OF REGENTS, THE UNIVERSITY OF TEXAS SYSTEM, Austin, TX (US)

(72) Inventor: Raghu KALLURI, Houston, TX (US)

(73) Assignee: BOARD OF REGENTS, THE UNIVERSITY OF TEXAS SYSTEM, Austin, TX (US)

(21) Appl. No.: 17/782,976

(22) PCT Filed: Dec. 4, 2020
(86) PCT No.: PCT/US2020/063475

§ 371 Date: Jun. 6, 2022

Related U.S. Application Data
(60) Provisional application No. 62/943,943, filed on Dec. 5, 2019.

(51) Int. Cl. A61K 9/51; C12N 15/113; A61P 1/16; A61K 31/14; A61K 31/045; A61K 31/047; A61K 31/4425; A61K 31/155; A61K 31/055; A61K 31/05; A61K 31/4178; A61K 31/31; A61K 31/713

(52) U.S. Cl. CPC A61K 9/5176; C12N 15/113; A61P 1/16; A61K 31/14; A61K 31/045; A61K 31/047; A61K 31/4425; A61K 31/155; A61K 31/055; A61K 31/05; A61K 31/4178; A61K 31/31; A61K 31/713; C12N 2310/14; C12N 2310/11; C12N 2320/32; C12N 2320/31

(57) ABSTRACT

Provided herein are compositions of lipid-based nanoparticles, such as exosomes, that contain a therapeutic STAT3-targeting inhibitory RNA. Also provided are methods of using such compositions to treat a patient having fibrosis or a disease associated with fibrosis.

### CROSS-REFERENCE TO RELATED APPLICATIONS

This application claims the benefit of priority of U.S. Provisional Patent Application No. 62/943,943 filed Dec. 5, 2019, which is hereby incorporated by reference in its entirety.

### BACKGROUND

### 1. Field

The present invention relates generally to the field of medicine. More particularly, it concerns compositions and methods for treating fibrosis and diseases associated with fibrosis.

### 2. Background

Liver fibrosis is characterized by excessive extracellular matrix (ECM) deposition in the liver, replacing the functional parenchyma and severely impacting health worldwide (Hernandez-Gea et al., 2001). Currently, there are no effective anti-fibrosis therapies, except for abating continued liver injury or liver transplantation (Bataller et al., 2005). Effective treatments for liver fibrosis urgently need innovative new approaches. Among the critical regulators of liver fibrosis, signal transducer and activator of transcription 3 (STAT3) signaling pathway is centrally implicated, driving the activation of fibroblasts and hepatic stellate cells (HSCs) and their conversion into myofibroblast-like phenotype (Chakraborty et al., 2017; Xiang et al., 2018; Pechkovsky et al., 2012). STAT3 is a transcription factor that is phosphorylated by Janus tyrosine kinases (JAK) in response to cytokine activation. Upon activation, the phosphorylated STAT3 dimerizes and translocates into the nucleus to activate the transcription of cytokine-responsive downstream genes (Chakraborty et al., 2017). Cytokines that activate STAT3 include TGFβ1 (Meng et al., 2016), IL-6 family of cytokines and growth hormone (GH). STAT3 activation has been reported in fibrotic liver observed in patients and mouse models (Xiang et al., 2018; Choi et al., 2019), and STAT3 inhibition using Sorafenib or other inhibitors partially ameliorates CC14-induced liver fibrosis in mice (Choi et al., 2019; Su et al., 2015). Although STAT3 has emerged as an important vulnerability for liver fibrosis, therapeutic targeting of STAT3 remains a challenge due to lack of STAT3 specific inhibitors (Bartneck et al., 2014). As such, new means of inhibiting STAT3 signaling are needed in order to develop specific anti-fibrotic therapies.

### SUMMARY

Embodiments of the disclosure include nanoparticles, compositions, pharmaceutical compositions, nucleic acids, inhibitory RNA molecules, methods for preparation of therapeutic compositions, methods for isolation of exosomes, methods for preparation of lipid-based nanoparticles, and methods for treatment of a subject. Compositions of the disclosure can include at least 1, 2, 3, 4, 5, or more of the following components: liposomes, exosomes, inhibitory RNA, siRNA, shRNA, miRNA, growth factors, unmodified antisense oligonucleotides, modified antisense oligonucleotides, and antimicrobial agents. In some embodiments, any one of more of these components may be excluded from a composition of the disclosure. Methods of the disclosure can include at least 1, 2, 3, 4, or more of the following steps: administering a pharmaceutical composition, administering an exosome, administering a liposome, administering an inhibitory RNA, generating a liposome, obtaining an exosome from a subject, purifying exosomes from mesenchymal cells, generating an inhibitory RNA, synthesizing an siRNA, preparing a lipid nanoparticle, introducing an inhibitory RNA into a lipid-based nanoparticle, encapsulating an inhibitory RNA in a nanoparticle, diagnosing a subject as having fibrosis, and treating a subject for fibrosis. It is contemplated that, in some embodiments, any one or more of these steps may be excluded from a method of the disclosure.

In some embodiments, provided herein are compositions comprising a lipid-based nanoparticle that contains an inhibitory RNA that hybridizes to a STAT3 polynucleotide. In some aspects, the lipid-based nanoparticle comprises CD47 on its surface. In some aspects, the lipid-based nanoparticle comprises a growth factor on its surface. In some aspects, the lipid-based nanoparticle is a liposome or an exosome. In some aspects, the inhibitory RNA is a siRNA, shRNA, antisense oligonucleotide, miRNA, or pre-miRNA. In certain aspects, the antisense oligonucleotide is modified. In some aspects, the inhibitory RNA knocks down the expression of STAT3 protein. In some aspects, the inhibitory RNA has a size between 18 and 30 nucleotides. In some embodiments, the inhibitory RNA comprises a sequence having at least, having at most, or having 80, 81, 82, 83, 84, 85, 86, 87, 88, 89, 90, 91, 92, 93, 94, 95, 96, 97, 98, 99, 99.5, 99.9, or 100% identity, or any range derivable therein, with any one of SEQ ID NOs:1-5. In some embodiments, the inhibitory RNA comprises a sequence having at least, having at most, or having 80, 81, 82, 83, 84, 85, 86, 87, 88, 89, 90, 91, 92, 93, 94, 95, 96, 97, 98, 99, 99.5, 99.9, or 100% identity, or any range derivable therein, with SEQ ID NO: 1. In some embodiments, the inhibitory RNA comprises SEQ ID NO:1. In some embodiments, the inhibitory RNA comprises a sequence having at least, having at most, or having 80, 81, 82, 83, 84, 85, 86, 87, 88, 89, 90, 91, 92, 93, 94, 95, 96, 97, 98, 99, 99.5, 99.9, or 100% identity, or any range derivable therein, with SEQ ID NO:2. In some embodiments, the inhibitory RNA comprises SEQ ID NO:2. In some embodiments, the inhibitory RNA comprises a sequence having at least, having at most, or having 80, 81, 82, 83, 84, 85, 86, 87, 88, 89, 90, 91, 92, 93, 94, 95, 96, 97, 98, 99, 99.5, 99.9, or 100% identity, or any range derivable therein, with SEQ ID NO:3. In some embodiments, the inhibitory RNA comprises SEQ ID NO:3. In some embodiments, the inhibitory RNA comprises a sequence having at least, having at most, or having 80, 81, 82, 83, 84, 85, 86, 87, 88, 89, 90, 91, 92, 93, 94, 95, 96, 97, 98, 99, 99.5, 99.9, or 100% identity, or any range derivable therein, with SEQ ID NO:4. In some embodiments, the inhibitory RNA comprises SEQ ID NO:4. In some embodiments, the inhibitory RNA comprises a sequence having at least, having at most, or having 80, 81, 82, 83, 84, 85, 86, 87, 88, 89, 90, 91, 92, 93, 94, 95, 96, 97, 98, 99, 99.5, 99.9, or 100% identity, or any range derivable therein, with SEQ ID NO:5. In some embodiments, the inhibitory RNA comprises SEQ ID NO:5. It is contemplated that, in some embodiments, any one or more of these components may be excluded from a composition of the disclosure. Also disclosed herein, in some embodiments, are methods of preparing therapeutic compositions comprising introducing an inhibitory RNA of the disclosure (e.g., an inhibitory RNA that hybridizes to a STAT3 polynucleotide) into a lipid-based nanoparticle (e.g., a liposome, an exosome, etc.).

In some embodiments, provided herein are pharmaceutical compositions comprising lipid-based nanoparticles of any one of the present embodiments and an excipient. In some aspects, the composition is formulated for parenteral administration. In certain aspects, the composition is formulated for intravenous, intramuscular, sub-cutaneous, or intraperitoneal injection. In certain aspects, the compositions further comprise an antimicrobial agent. In certain aspects, the antimicrobial agent is benzalkonium chloride, benzethonium chloride, benzyl alcohol, bronopol, centrimide, cetylpyridinium chloride, chlorhexidine, chlorobutanol, chlorocresol, chloroxylenol, cresol, ethyl alcohol, glycerin, exetidine, imidurea, phenol, phenoxyethanol, phenylethl alcohol, phenlymercuric nitrate, propylene glycol, or thimerosal.

In some embodiments, provided herein are methods of treating fibrosis or a condition associated with fibrosis in a patient in need thereof comprising administering a composition of any one of the present embodiments to the patient. In some aspects, administering the pharmaceutical composition results in delivery of the inhibitory RNA to a cell in the patient. In some aspects, the fibrosis is liver fibrosis, lung fibrosis, pulmonary fibrosis, cystic fibrosis, idiopathic pulmonary fibrosis (IPF), or radiation-induced lung injury. In some embodiments, the fibrosis is liver fibrosis. In some aspects, the pharmaceutical composition is administered via systemic administration. In certain aspects, the systemic administration is intravenous administration. In certain aspects, the methods further comprise administering at least a second therapy to the patient. In some aspects, the patient is a human. In certain aspects, the lipid-based nanoparticles are exosomes, wherein the exosomes are autologous to the patient. In certain aspects, the exosomes are obtained from a body fluid sample obtained from the patient. In certain aspects, the body fluid sample is blood, lymph, saliva, urine, cerebrospinal fluid, bone marrow aspirates, eye exudate/tears, or serum. In certain aspects, the exosomes are obtained from a mesenchymal cell. In certain aspects, the composition is administered more than once. In some embodiments, the composition is administered at least or at most 2, 3, 4, 5, 6, 7, 8, 9, 10, 11, 12, 13, 14, or 15 times, or any range derivable therein. In certain aspects, administering a composition of the disclosure (e.g., a composition comprising a lipid-based nanoparticle that contains an inhibitory RNA that hybridizes to a STAT3 polynucleotide) reduces expression of one or more STAT3-associated genes in cells (e.g., hepatic cells) of a patient. In some embodiments, administering the composition reduces expression of Col1a1 in hepatic cells of the patient. In some embodiments, administering the composition reduces expression of Acta2 in hepatic cells of the patient. In some embodiments, administering the composition reduces expression of Col1a2 in hepatic cells of the patient. In some embodiments, administering the composition reduces expression of Vim in hepatic cells of the patient.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, such as below 0.01%. In some embodiments, a composition “essentially free” of a specified component contains or contains at most 0.05%, 0.04%, 0.03%, 0.02%, 0.01%, 0.005%, 0.001%, 0.0001%, or less of the specified component. In some embodiments, a composition “essentially free” of a specified component is one in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the measurement or quantitation method.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments are discussed throughout this application. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. The embodiments in the Example section are understood to be embodiments that are applicable to all aspects of the technology described herein.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Liver IVIS imaging.

FIGS. 2A-2B. Damage of Liver Parenchyma. FIG. 2A shows H&E-stained sections of liver from fibrotic mice treated with various exosomes treatments. FIG. 2B shows a quantification of the level of fibrosis seen in FIG. 2A.

FIGS. 3A-3B. Damage of Lung Parenchyma. FIG. 3A shows H&E-stained sections of lungs from fibrotis mice treated with various exosomes treatments. FIG. 3B shows a quantification of the level of fibrosis seen in FIG. 3A.

FIGS. 4A-4F. Knockdown efficiency of STAT3 in primary HSCs and biodistribution of exosomes. FIGS. 4A and 4B show relative Stat3 expression in HSC treated with 5 μg/2 billion iExo^{siRNA-STAT3} (FIG. 4A) or iExo^{mASO-STAT3} (FIG. 4B). FIG. 4C shows representative images of the listed organs analyzed for presence of DiR-labeled exosomes in non-fibrotic (sham) (left panel) and fibrotic mice (right panel) (n=1). FIGS. 4D-4F show immunofluorescence imaging (FIG. 4D) and quantification (FIGS. 4E and 4F) of AF647 labeled exosomes and DAPI in frozen liver tissues of the listed groups (3 visual fields for each tissue analyzed). Scale bar: 100 μm. Data are represented as mean±SEM. For FIGS. 4A and 4D, an unpaired two-tailed t-test was used. For FIG. 4B, a one-way ANOVA with Sidak's post-hoc analysis was used. *p<0.05, **p<0.01, ***p<0.001, ****p<0.0001.

FIGS. 5A-5K. FIG. 5A shows images of mouse hepatic stellate cells (HSCs) cultured for 7 days (left panel). Scale bar: 100 μm. Immunofluorescence staining for α-SMA and DAPI of primary mouse HSCs (center and right panels). Scale bar: 100 μm. FIGS. 5B and 5C show qPCR analysis of STAT3. FIG. 5D shows a schematic of CCl₄ and iExosomes/siRNA/mASO treatment schedule. Upper arrows indicate CCl₄ injections (Day 0), and lower arrows indicate iExosomes/siRNA/mASO injections (Day 9). FIGS. 5E and 5F show immunohistochemical staining of Collagen I (FIG. 5E) and quantification (FIG. 5F) in mice treated with 1 μg of 1 billion iExo^{siRNA-STAT3} or iExo^{mASO-STAT3}. (3 visual fields for each tissue analyzed). n=3; Scale bar: 100 inn. FIGS. 5G and 5H show immunofluorescence staining of α-SMA (FIG. 5G) and quantification (FIG. 5H) in mice treated with 1 μg of 1 billion iExo^{siRNA-STAT3} or iExo^{mASO-STAT3}. (3 visual fields for each tissue analyzed). n=3. Scale bar: 100 μm. FIG. 5I shows H&E staining of liver from mice treated with 1 μg of 1 billion iExo^{siRNA STAT3} or iExo^{mASO-STAT3} (5 visual fields for each tissue analyzed), n=5; Scale bar: 100 μm. FIGS. 5J and 5K show percentage of necrotic (FIG. 5J) and degenerated hepatocytes (FIG. 5K). The data is presented as mean±SEM. Individual dots in graphs depict distinct mice. FIG. 5B (left panel), unpaired two-tailed Student's t-test. FIG. 5B (right panel) and FIGS. 5C-5K One-way ANOVA with Sidak's post-hoc analysis; p values are indicated in all of the graphs. *p<0.05; **p<0.01; ****p<0.0001; ns: not significant.

FIGS. 6A-6J. iExosomes targeting STAT3 reduced liver fibrosis. FIGS. 6A and 6B show relative mRNA expression of STAT3 in liver of mice treated with 1 μg/l billion (FIG. 6A) or 5 μg/2 billion (FIG. 6B) iExo^{siRNA-STAT3} or iExo^{mASO-STAT3} of the indicated treatments. n=4-5 distinct mice in 5 μg/2 billion groups; n=4-5 distinct mice, one-way ANOVA was used in 1 μg/l billion group. FIGS. 6C and 6D show representative Sirius red staining (FIG. 6C) and quantification (FIG. 6D) of liver sections from the 1 μg/l billion treatment group (3 visual fields for each tissue analyzed). n=5-6 distinct mice, One-way ANOVA. Scale bar: 100 μm. The graph depicts the percent Sirius red positive area. FIGS. 6E and 6F show representative Sirius red staining (FIG. 6E) and quantification (FIG. 6F) of liver sections from the 5 μg/2 billion treatment group (3 visual fields for each tissue analyzed). n=3-5 distinct mice. Scale bar: 100 μm. The graph depicts the percent Sirius red positive area. FIGS. 6G and 6H show representative images (3 visual fields for each tissue analyzed) of immunohistochemical staining for Collagen I (FIG. 6G) and quantification of the percent of Collagen r area per visual field (100×) (FIG. 6H). n=3. FIGS. 6I and 6J show α-SMA immunofluorescence staining (FIG. 6I; 3 visual fields for each tissue analyzed) and quantification (FIG. 6J) of the number of α-SMA⁺ cells per visual field (100×). n=3-4 distinct mice; Scale bar: 100 μm. The data are presented as mean±SEM. Individual dots in graphs depict distinct mice. One-way ANOVA or 2-tailed unpaired t test, unless otherwise indicated; p values are indicated in all of the graphs. *p<0.05; **p<0.01; ***p<0.001; ****p<0.0001; ns: not significant.

FIGS. 7A-7K. iExosomes targeting STAT3 preserved liver functional parenchyma. FIGS. 7A-7D show relative Col1a1 (FIGS. 7A and 7B) and Acta2 (FIGS. 7C and 7D) expression in livers with the indicated treatments. n=4-5 distinct mice in 5 μg/2 billion group; n=5 distinct mice in 1 μg/l billion groups, one-way ANOVA was used for statistical analysis in 1 μg/l billion group. FIGS. 7E and 7F show serum levels of ALT in mice with the indicated treatments. n=4-5 distinct mice in 5 μg/2 billion groups; n=4-5 distinct mice, one-way ANOVA was used for statistical analysis in 1 μg/l billion group.

FIGS. 7G and 7H show serum levels of AST in mice with the indicated treatments. n=4-5 distinct mice in 5 μg/2 billion group; n=4-5 distinct mice in 1 μg/l billion groups, one-way ANOVA was used for statistical analysis in 1 μg/l billion group. FIG. 7I shows H&E staining of paraffin-embedded liver sections (3-5 visual fields for each tissue analyzed). n=4-5 distinct mice; Scale bar: 100 μm. FIGS. 7J and 7K show percentage of necrotic and degenerated hepatocytes. The data is presented as mean±SEM. Individual dots in graphs depict distinct mice. One-way ANOVA or unpaired two-tailed t-test; p values are indicated in all of the graphs. *p<0.05; **p<0.01; ****p<0.0001; ns: not significant.

FIGS. 8A and 8B. FIG. 8A shows H&E staining of the listed organs in mice treated with 5 μg/2 billion iExo^{siRNA-STAT3} or iExo^{mASO-STAT3}. FIG. 8B shows H&E staining of the listed organs in mice treated with 1 μg/1 billion iExo^{siRNA-STAT3} or iExo^{mASO-STAT3}.

FIGS. 9A-9H. Reprogramming of the fibrotic liver transcriptome during iExosomes treatment. FIG. 9A shows a heat map depicting relative intensity of all probes amongst the experimental groups (siCntrl iExo (n=3), siSTAT3 iExo (n=3), mASO Scrbl iExo (n=3) and mASO STAT3 iExo (n=3). Euclidean clustering of both rows and columns using log₂-transformed mRNA-Seq expression data. FIGS. 9B and 9C show volcano plots depicting the number of differentially regulated genes in the livers of the listed experimental groups. FIG. 9D shows a heat map of STAT3 signaling. FIG. 9E shows selected genes associated with ECM deposition and remodeling. FIGS. 9F and 9G show a representation of differences in target genes by using gene ontology (GO) analysis (WebGestalt) enrichment.

FIG. 9H shows an interaction network generated by the NetworkAnalyst for the STAT3 signaling and ECM-associated genes.

FIGS. 10A-10H. Col1a1 knockout in activated hepatic stellate cells. FIG. 10A shows Sirius Red staining to assess ECM and collagen I associated fibrosis, demonstrating significant decrease upon genetic loss of type I collagen from activated hepatic stellate cells or αSMA+ myofibroblasts)(Col1a1^{cKO} in the context of fibrosis. FIG. 10B shows results demonstrating a significant reduction of Collagen I in Col1a1^{cKO} with liver fibrosis. FIG. 10C shows results demonstrating a significant improvement in liver histology in Col1a1^{cKO} with liver fibrosis. FIGS. 10D-10F show quantitation of the results from FIGS. 10A-10C. FIGS. 10G and 10H show gene expression data demonstrating that many of the global expression patterns associated with liver fibrosis are significantly improved in Col1a1^{cKO} mice with liver fibrosis.

### DETAILED DESCRIPTION

Provided herein are exosomes that have been engineered to carry inhibitory RNA molecules, including anti-sense oligonucleotides (ASO) and siRNA, targeting STAT3, a mediator of organ fibrosis, including liver and lung fibrosis. These engineered exosomes can be used to treat fibrosis, including liver fibrosis and lung fibrosis. Since exosomes obtained from mesenchymal stem cells have very high distribution to the lung and the liver, the delivery of the ASO or siRNA is efficient.

### I. LIPID-BASED NANOPARTICLES

A lipid-based nanoparticle may be a liposome, an exosome, a lipid preparation, or another lipid-based nanoparticle, such as a lipid-based vesicle (e.g., a DOTAP:cholesterol vesicle). Lipid-based nanoparticles may be positively charged, negatively charged, or neutral. Lipid-based nanoparticles may comprise the necessary components to allow for transcription and translation, signal transduction, chemotaxis, or other cellular functions. It is contemplated that one or more of these items may be excluded in an embodiment.

Lipid-based nanoparticles may comprise CD47 on their surface. CD47 (Integrin Associated Protein) is a transmembrane protein that is expressed on most tissues and cells. CD47 is a ligand for Signal Regulatory Protein Alpha (SIRP-α), which is expressed on phagocytic cells such as macrophages and dendritic cells. Activated CD47-SIRP-α initiates a signal transduction cascade that inhibits phagocytosis. Thus, without being bound by theory, expression of CD47 on the surface of exosomes may prevent phagocytosis by macrophages (see WO 2016/201323, which is incorporated herein by reference in its entirety).

A. Liposomes

A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition. Liposomes provided herein include unilamellar liposomes, multilamellar liposomes, and multivesicular liposomes. Liposomes provided herein may be positively charged, negatively charged, or neutrally charged. In certain embodiments, the liposomes are neutral in charge.

A multilamellar liposome has multiple lipid layers separated by aqueous medium. Such liposomes form spontaneously when lipids comprising phospholipids are suspended in an excess of aqueous solution. The lipid components undergo self-rearrangement before the formation of closed structures and entrap water and dissolved solutes between the lipid bilayers. Lipophilic molecules or molecules with lipophilic regions may also dissolve in or associate with the lipid bilayer.

In specific aspects, a polypeptide, a nucleic acid, or a small molecule drug may be, for example, encapsulated in the aqueous interior of a liposome, interspersed within the lipid bilayer of a liposome, attached to a liposome via a linking molecule that is associated with both the liposome and the polypeptide/nucleic acid, entrapped in a liposome, complexed with a liposome, or the like.

A liposome used according to the present embodiments can be made by different methods, as would be known to one of ordinary skill in the art. For example, a phospholipid, such as for example the neutral phospholipid dioleoylphosphatidylcholine (DOPC), is dissolved in tert-butanol. The lipid(s) is then mixed with a polypeptide, nucleic acid, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min to 2 h, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

The dried lipids or lyophilized liposomes prepared as described above may be dehydrated and reconstituted in a solution of a protein or peptide and diluted to an appropriate concentration with a suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000×g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

Additional liposomes which may be useful with the present embodiments include cationic liposomes, for example, as described in WO02/100435A1, U.S. Pat. No. 5,962,016, U.S. Application 2004/0208921, WO03/015757A1, WO04/029213A2, U.S. Pat. Nos. 5,030,453, and 6,680,068, all of which are hereby incorporated by reference in their entirety without disclaimer.

In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040, each incorporated herein by reference.

In certain embodiments, the lipid-based nanoparticle is a neutral liposome (e.g., a DOPC liposome). “Neutral liposomes” or “non-charged liposomes”, as used herein, are defined as liposomes having one or more lipid components that yield an essentially-neutral, net charge (substantially non-charged). By “essentially neutral” or “essentially non-charged”, it is meant that few, if any, lipid components within a given population (e.g., a population of liposomes) include a charge that is not canceled by an opposite charge of another component (i.e., fewer than 10% of components include a non-canceled charge, more preferably fewer than 5%, and most preferably fewer than 1%). In certain embodiments, neutral liposomes may include mostly lipids and/or phospholipids that are themselves neutral under physiological conditions (i.e., at about pH 7).

Liposomes and/or lipid-based nanoparticles of the present embodiments may comprise a phospholipid. In certain embodiments, a single kind of phospholipid may be used in the creation of liposomes (e.g., a neutral phospholipid, such as DOPC, may be used to generate neutral liposomes). In other embodiments, more than one kind of phospholipid may be used to create liposomes. Phospholipids may be from natural or synthetic sources. Phospholipids include, for example, phosphatidylcholines, phosphatidylglycerols, and phosphatidylethanolamines; because phosphatidylethanolamines and phosphatidyl cholines are non-charged under physiological conditions (i.e., at about pH 7), these compounds may be particularly useful for generating neutral liposomes. In certain embodiments, the phospholipid DOPC is used to produce non-charged liposomes. In certain embodiments, a lipid that is not a phospholipid (e.g., a cholesterol) may be used.

Phospholipids include glycerophospholipids and certain sphingolipids. Phospholipids include, but are not limited to, dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), distearoylsphingomyelin (“DS SP”), distearoylphophatidylethanolamine (“DSPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, and dilinoleoylphosphatidylcholine.

B. Exosomes

The terms “microvesicle” and “exosomes,” as used herein, refer to a membranous particle having a diameter (or largest dimension where the particles is not spheroid) of between about 10 nm to about 5000 nm, more typically between 30 nm and 1000 nm, and most typically between about 50 nm and 750 nm, wherein at least part of the membrane of the exosomes is directly obtained from a cell. An exosome of the disclosure may have a diameter of at least, at most, or about 10, 20, 30, 40, 50, 60, 70, 80, 90, 100, 200, 300, 400, 500, 600, 700, 800, 900, 1000, 2000, 3000, 4000, or 5000 nm, or any range derivable therein. Most commonly, exosomes will have a size (average diameter) that is up to 5% of the size of the donor cell. Therefore, especially contemplated exosomes include those that are shed from a cell.

Exosomes may be detected in or isolated from any suitable sample type, such as, for example, body fluids. As used herein, the term “isolated” refers to separation out of its natural environment and is meant to include at least partial purification and may include substantial purification. As used herein, the term “sample” refers to any sample suitable for the methods provided by the present invention. The sample may be any sample that includes exosomes suitable for detection or isolation. Sources of samples include blood, bone marrow, pleural fluid, peritoneal fluid, cerebrospinal fluid, urine, saliva, amniotic fluid, malignant ascites, broncho-alveolar lavage fluid, synovial fluid, breast milk, sweat, tears, joint fluid, and bronchial washes. In one aspect, the sample is a blood sample, including, for example, whole blood or any fraction or component thereof. A blood sample suitable for use with the present invention may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with cancer.

Exosomes may also be isolated from tissue samples, such as surgical samples, biopsy samples, tissues, feces, and cultured cells. When isolating exosomes from tissue sources it may be necessary to homogenize the tissue in order to obtain a single cell suspension followed by lysis of the cells to release the exosomes. When isolating exosomes from tissue samples it is important to select homogenization and lysis procedures that do not result in disruption of the exosomes. Exosomes contemplated herein are preferably isolated from body fluid in a physiologically acceptable solution, for example, buffered saline, growth medium, various aqueous medium, etc.

Exosomes may be isolated from freshly collected samples or from samples that have been stored frozen or refrigerated. In some embodiments, exosomes may be isolated from cell culture medium. Although not necessary, higher purity exosomes may be obtained if fluid samples are clarified before precipitation with a volume-excluding polymer, to remove any debris from the sample. Methods of clarification include centrifugation, ultracentrifugation, filtration, or ultrafiltration. Most typically, exosomes can be isolated by numerous methods well-known in the art. One preferred method is differential centrifugation from body fluids or cell culture supernatants. Exemplary methods for isolation of exosomes are described in (Losche et al., 2004; Mesri and Altieri, 1998; Morel et al., 2004). Alternatively, exosomes may also be isolated via flow cytometry as described in (Combes et al., 1997).

One accepted protocol for isolation of exosomes includes ultracentrifugation, often in combination with sucrose density gradients or sucrose cushions to float the relatively low-density exosomes. Isolation of exosomes by sequential differential centrifugations is complicated by the possibility of overlapping size distributions with other microvesicles or macromolecular complexes. Furthermore, centrifugation may provide insufficient means to separate vesicles based on their sizes. However, sequential centrifugations, when combined with sucrose gradient ultracentrifugation, can provide high enrichment of exosomes.

Isolation of exosomes based on size, using alternatives to the ultracentrifugation routes, is another option. Successful purification of exosomes using ultrafiltration procedures that are less time consuming than ultracentrifugation, and do not require use of special equipment have been reported. Similarly, a commercial kit is available (EXOMIR™, Bioo Scientific) which allows removal of cells, platelets, and cellular debris on one microfilter and capturing of vesicles bigger than 30 nm on a second microfilter using positive pressure to drive the fluid. However, for this process, the exosomes are not recovered, their RNA content is directly extracted from the material caught on the second microfilter, which can then be used for PCR analysis. HPLC-based protocols could potentially allow one to obtain highly pure exosomes, though these processes require dedicated equipment and are difficult to scale up. A significant problem is that both blood and cell culture media contain large numbers of nanoparticles (some non-vesicular) in the same size range as exosomes. For example, some miRNAs may be contained within extracellular protein complexes rather than exosomes; however, treatment with protease (e.g., proteinase K) can be performed to eliminate any possible contamination with “extraexosomal” protein.

In another embodiment, cancer cell-derived exosomes may be captured by techniques commonly used to enrich a sample for exosomes, such as those involving immunospecific interactions (e.g., immunomagnetic capture). Immunomagnetic capture, also known as immunomagnetic cell separation, typically involves attaching antibodies directed to proteins found on a particular cell type to small paramagnetic beads. When the antibody-coated beads are mixed with a sample, such as blood, they attach to and surround the particular cell. The sample is then placed in a strong magnetic field, causing the beads to pellet to one side. After removing the blood, captured cells are retained with the beads. Many variations of this general method are well-known in the art and suitable for use to isolate exosomes. In one example, the exosomes may be attached to magnetic beads (e.g., aldehyde/sulphate beads) and then an antibody is added to the mixture to recognize an epitope on the surface of the exosomes that are attached to the beads. Exemplary proteins that are known to be found on cancer cell-derived exosomes include ATP-binding cassette sub-family A member 6 (ABCA6), tetraspanin-4 (TSPAN4), SLIT and NTRK-like protein 4 (SLITRK4), putative protocadherin beta-18 (PCDHB18), myeloid cell surface antigen CD33 (CD33), and glypican-1 (GPC1). Cancer cell-derived exosomes may be isolated using, for example, antibodies or aptamers to one or more of these proteins.

It should be noted that not all proteins expressed in a cell are found in exosomes secreted by that cell. For example, calnexin, GM130, and LAMP-2 are all proteins expressed in MCF-7 cells but not found in exosomes secreted by MCF-7 cells (Baietti et al., 2012). As another example, one study found that 190/190 pancreatic ductal adenocarcinoma patients had higher levels of GPC1+ exosomes than healthy controls (Melo et al., 2015, which is incorporated herein by reference in its entirety). Notably, only 2.3% of healthy controls, on average, had GPC1+ exosomes.

1. Exemplary Protocol for Collecting Exosomes from Cell Culture

On Day 1, seed enough cells (e.g., about five million cells) in T225 flasks in media containing 10% FBS so that the next day the cells will be about 70% confluent. On Day 2, aspirate the media on the cells, wash the cells twice with PBS, and then add 25-30 mL base media (i.e., no PenStrep or FBS) to the cells. Incubate the cells for 24-48 hours. A 48 hour incubation is preferred, but some cells lines are more sensitive to serum-free media and so the incubation time should be reduced to 24 hours. Note that FBS contains exosomes that will heavily skew NanoSight results.

On Day 3/4, collect the media and centrifuge at room temperature for five minutes at 800×g to pellet dead cells and large debris. Transfer the supernatant to new conical tubes and centrifuge the media again for 10 minutes at 2000×g to remove other large debris and large vesicles. Pass the media through a 0.2 μm filter and then aliquot into ultracentrifuge tubes (e.g., 25×89 mm Beckman Ultra-Clear) using 35 mL per tube. If the volume of media per tube is less than 35 mL, fill the remainder of the tube with PBS to reach 35 mL. Ultracentrifuge the media for 2-4 hours at 28,000 rpm at 4° C. using a SW 32 Ti rotor (k-factor 266.7, RCF max 133,907). Carefully aspirate the supernatant until there is roughly 1-inch of liquid remaining. Tilt the tube and allow remaining media to slowly enter aspirator pipette. If desired, the exosomes pellet can be resuspended in PBS and the ultracentrifugation at 28,000 rpm repeated for 1-2 hours to further purify the population of exosomes.

Finally, resuspend the exosomes pellet in 210 μL PBS. If there are multiple ultracentrifuge tubes for each sample, use the same 210 μL PBS to serially resuspend each exosomes pellet. For each sample, take 10 μL and add to 990 μL H₂O to use for nanoparticle tracking analysis. Use the remaining 200 μL exosomes-containing suspension for downstream processes or immediately store at −80° C.

2. Exemplary Protocol for Extracting Exosomes from Serum Samples

First, allow serum samples to thaw on ice. Then, dilute 250 μL of cell-free serum samples in 11 mL PBS; filter through a 0.2 μm pore filter. Ultracentrifuge the diluted sample at 150,000×g overnight at 4° C. The following day, carefully discard the supernatant and wash the exosomes pellet in 11 mL PBS. Perform a second round of ultracentrifugation at 150,000×g at 4° C. for 2 hours. Finally, carefully discard the supernatant and resuspend the exosomes pellet in 100 μL PBS for analysis.

C. Exemplary Protocol for Electroporation of Exosomes and Liposomes

Mix 1×10⁸ exosomes (measured by NanoSight analysis) or 100 nm liposomes (e.g., purchased from Encapsula Nano Sciences) and 1 μg of siRNA (Qiagen) or shRNA in 400 μL of electroporation buffer (1.15 mM potassium phosphate, pH 7.2, 25 mM potassium chloride, 21% Optiprep). Electroporate the exosomes or liposomes using a 4 mm cuvette (see, e.g., Alvarez-Erviti et al., 2011; El-Andaloussi et al., 2012). After electroporation, treat the exosomes or liposomes with protease-free RNAse followed by addition of 10× concentrated RNase inhibitor. Finally, wash the exosomes or liposomes with PBS under ultracentrifugation methods, as described above.

### II. INHIBITORY RNAS

A. Antisense Oligonucleotides

Antisense oligonucleotide (ASO) therapeutic agents are single stranded nucleic acid therapeutics, typically about 16 to 30 nucleotides in length, and are complementary to a target nucleic acid sequence in the target cell, either in culture or in an organism.

In some embodiments, the agent is a single-stranded antisense RNA molecule, a single-stranded antisense DNA molecule, or a single-stranded antisense polynucleotide comprising both DNA and RNA. In a particular embodiment, the antisense molecule is an ASO comprising both DNA and RNA. An antisense molecule is complementary to a sequence within the target mRNA, e.g., a STAT3 mRNA. Antisense molecules can inhibit translation in a stoichiometric manner by base pairing to the mRNA and physically obstructing the translation machinery. The antisense molecule may have at least or at most 15-30 nucleotides that are complementary to the target mRNA. For example, the antisense molecule may have a sequence of at least or at most 15, 16, 17, 18, 19, 20, 21, 22, 23, 24 or 25, or any range or value derivable therein, contiguous nucleotides that are complementary to the target mRNA.

In some embodiments, the ASO comprises at least or at most 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides, or any range or value derivable therein. Any of these values may be used to define a range for the number of nucleotides in the ASO. For example, the ASO may comprise, comprise at least or, or comprise at most 8-50, 15-30, or 20-25 nucleotides. In some embodiments, the ASO consists of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides, or any range or value derivable therein. Any of these values may be used to define a range for the number of nucleotides in the ASO. For example, the ASO may consist of 8-50, 15-30, or 20-25 nucleotides.

In one aspect of the disclosure, the agent is a single-stranded antisense nucleic acid molecule (ASO). Antisense oligonucleotides (ASOs) are synthetic molecules and, in some embodiments, comprise between 18-21 nucleotides in length and are complementary to the mRNA sequence of the target gene. ASOs bind cognate mRNA sequences through sequence-specific hybridization resulting in cleavage or disablement of the mRNA and inhibition of the expression of the target gene.

1. Modification of ASOs

In certain embodiments, the ASOs of the disclosure may be modified. A “modified ASO” refers to a molecule in which one or more of the components of the nucleic acid, namely sugars, bases, and phosphate moieties, are different from that which occur in nature, for example, different from that which occurs in the human body. Several modifications to ASOs are described in the art. These modifications are aimed at improving ASO properties such as resistance to nucleases, permeability across biological membranes, solubility, stability, or modulation of pharmacokinetic and pharmacodynamics properties while maintaining specificity to the target mRNA. For example, the modifications on the nucleotides can include, but are not limited to, LNA, HNA, CeNA, 2′-methoxyethyl, 2′-O-alkyl, 2′-O-allyl, 2′-C-allyl, 2′-fluoro, 2′-deoxy, 2′-hydroxyl, and combinations thereof. It is contemplated that one or more of these modifications may be excluded in an embodiment.

Patents directed to antisense nucleic acids, chemical modifications, and therapeutic uses are provided, for example, in U.S. Pat. No. 5,898,031 related to chemically modified RNA-containing therapeutic compounds, and U.S. Pat. No. 6,107,094 related methods of using these compounds as therapeutic agent. U.S. Pat. No. 7,432,250 related to methods of treating patients by administering single-stranded chemically modified RNA-like compounds; and U.S. Pat. No. 7,432,249 related to pharmaceutical compositions containing single-stranded chemically modified RNA-like compounds. U.S. Pat. No. 7,629,321 is related to methods of cleaving target mRNA using a single-stranded oligonucleotide having a plurality RNA nucleosides and at least one chemical modification. Each of the patents listed in this paragraph are incorporated herein by reference in their entirety.

a. Modified Bases

Therapeutic nucleic acid may include natural (i.e. A, G, U, C, or T) or modified (e.g. 7-deazaguanosine, inosine, etc.) bases. Modification of bases includes the incorporation of modified bases (or modified nucleoside or modified nucleotides) that are variations of standard bases, sugars and/or phosphate backbone chemical structures occurring in ribonucleic (i.e., A, C, G and U) and deoxyribonucleic (i.e., A, C, G and T) acids. Included within this scope are, for example: Gm (2′-methoxyguanylic acid), Am (2′-methoxyadenylic acid), Cf (2′-fluorocytidylic acid), Uf (2′-fluorouridylic acid), Ar (riboadenylic acid). The aptamers may also include cytosine or any cytosine-related base including 5-methylcytosine, 4-acetylcytosine, 3-methylcytosine, 5-hydroxymethyl cytosine, 2-thiocytosine, 5-halocytosine (e.g., 5-fluorocytosine, 5-bromocytosine, 5-chlorocytosine, and 5-iodocytosine), 5-propynyl cytosine, 6-azocytosine, 5-trifluoromethylcytosine, N4,N4-ethanocytosine, phenoxazine cytidine, phenothiazine cytidine, carbazole cytidine or pyridoindole cytidine. The aptamer may further include guanine or any guanine-related base including 6-methylguanine, 1-methylguanine, 2,2-dimethylguanine, 2-methylguanine, 7-methylguanine, 2-propylguanine, 6-propylguanine, 8-haloguanine (e.g., 8-fluoroguanine, 8-bromoguanine, 8-chloroguanine, and 8-iodoguanine), 8-aminoguanine, 8-sulfhydrylguanine, 8-thioalkylguanine, 8-hydroxylguanine, 7-methylguanine, 8-azaguanine, 7-deazaguanine or 3-deazaguanine. The aptamer may still further include adenine or any adenine-related base including 6-methyladenine, N6-isopentenyladenine, N6-methyladenine, 1-methyladenine, 2-methyladenine, 2-methylthio-N6-isopentenyladenine, 8-haloadenine (e.g., 8-fluoroadenine, 8-bromoadenine, 8-chloroadenine, and 8-iodoadenine), 8-aminoadenine, 8-sulfhydryladenine, 8-thioalkyladenine, 8-hydroxyladenine, 7-methyladenine, 2-haloadenine (e.g., 2-fluoroadenine, 2-bromoadenine, 2-chloroadenine, and 2-iodoadenine), 2-aminoadenine, 8-azaadenine, 7-deazaadenine or 3-deazaadenine. Also included are uracil or any uracil-related base including 5-halouracil (e.g., 5-fluorouracil, 5-bromouracil, 5-chlorouracil, 5-iodouracil), 5-(carboxyhydroxylmethyl)uracil, 5-carboxymethylaminomethyl-2-thiouracil, 5-carboxymethylaminomethyluracil, dihydrouracil, 1-methylpseudouracil, 5-methoxyaminomethyl-2-thiouracil, 5′-methoxycarbonylmethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid, pseudouracil, 5-methyl-2-thiouracil, 2-thiouracil, 3-(3-amino-3-N-2-carboxypropyl)uracil, 5-methylaminomethyluracil, 5-propynyl uracil, 6-azouracil, or 4-thiouracil.

Examples of other modified base variants known in the art include, without limitation, e.g., 4-acetylcytidine, 5-(carboxyhydroxylmethyl)uridine, 2′-methoxycytidine, 5-carboxymethylaminomethyl-2-thioridine, 5-carboxymethylaminomethyluridine, dihydrouridine, 2′-O-methylpseudouridine, b-D-galactosylqueosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, b-D-mannosylqueosine, 5-methoxycarbonylmethyluridine, 5-methoxyuridine, 2-methylthio-N6-isopentenyladenosine, N-((9-b-D-ribofuranosyl-2-methylthiopurine-6-yl)carbamoyl)threonine, N-((9-b-D-ribofuranosylpurine-6-yl)N-methyl-carbamoyl)threonine, urdine-5-oxyacetic acid methylester, uridine-5-oxyacetic acid (v), wybutoxosine, pseudouridine, queosine, 2-thiocytidine, 5-methyl-2-thiouridine, 2-thiouridine, 4-thiouridine, 5-methyluridine, N-((9-b-D-ribofuranosylpurine-6-yl)carbamoyl)threonine, 2′-O-methyl-5-methyluridine, 2′-O-methyluridine, and wybutosine, 3-(3-amino-3-carboxypropyl)uridine.

Also included are the modified nucleobases described in U.S. Pat. Nos. 3,687,808, 3,687,808, 4,845,205, 5,130,302, 5,134,066, 5,175,273, 5,367,066, 5,432,272, 5,457,187, 5,459,255, 5,484,908, 5,502,177, 5,525,711, 5,552,540, 5,587,469, 5,594,121, 5,596,091, 5,614,617, 5,645,985, 5,830,653, 5,763,588, 6,005,096, and 5,681,941, each of which is incorporated herein by reference in its entirety.

b. Modified Sugars

Modified sugar moieties for use in ASOs are well known in the art and are described for example in U.S. Pat. No. 9,045,754 which is incorporated by reference herein in its entirety. Modified sugars can be used to alter, typically increase, the affinity of the ASO for its target and/or increase nuclease resistance. For example, in some embodiments, the binding affinity of the ASOs to their target can be increased by incorporating substituent groups in the nucleoside subunits of the ASOs. In some embodiments, the substituent groups are T substituent groups, substituent groups located at the 2′ position of the pentofuranosyl sugar moieties of the nucleoside subunits of the ASOs. Substituent groups include, but are not limited to, fluoro, alkoxy, amino-alkoxy, allyloxy, imidazolylalkoxy and polyethylene glycol. Alkoxy and aminoalkoxy groups generally include lower alkyl groups, particularly C1-C9 alkyl. In a particular embodiment, the 2′ substituent group is 2′-O-methyl. Polyethylene glycols are of the structure (O—CH2-CH2)n-O-alkyl. In a particular embodiment, the substituent is a polyethylene glycol substituent of the formula (—O—CH2-CH2)n-O-alkyl, wherein n=1 and alkyl=CH3. This modification has been shown to increase both affinity of an oligonucleotide for its target and nuclease resistance of an oligonucleotide. See U.S. Pat. No. 7,629,321 cited above. A further particularly useful 2′-substituent group for increasing the binding affinity is the 2′-fluoro group.

Examples of modified nucleoside and nucleotide sugar backbone variants known in the art include, without limitation, those having, e.g., 2′ ribosyl substituents such as F, SH, SCH3, OCN, Cl, Br, CN, CF₃, OCF3, SOCH3, SO2, CH3, ONO2, NO2, N3, NH2, OCH2CH2OCH3, O(CH2)2ON(CH3)2, OCH2OCH2N(CH3)2, O(C1-10 alkyl), O(C2-10 alkenyl), O(C2-10 alkynyl), S(C1-10 alkyl), S(C2-10 alkenyl), S(C2-10 alkynyl), NH(C1-10 alkyl), NH(C2-10 alkenyl), NH(C2-10 alkynyl), and O-alkyl-O-alkyl. Desirable 2′ ribosyl substituents include 2′-methoxy (2′-OCH3), 2′-aminopropoxy (2′ OCH2CH2CH2NH2), 2′-O-allyl (2′-CH2-CH═CH2), 2′-O-allyl (2′-O—CH2-CH═CH2), 2′-amino (2′-NH2), and 2′-fluoro (2′-F). The 2′-substituent may be in the arabino (up) position or ribo (down) position. One or more of these variants may be excluded from embodiments of the disclosure.

Another class of modified ASOs known in the art and that may be utilized in the ASOs of the disclosure contain alkyl modifications at the 2′ position of the ribose moiety. These ASOs were developed to improve the binding affinity and hybridization stability with target mRNA, and to increase the nuclease resistance of the ASOs. In this category, the most commonly used ASOs are 2′-O-Methyl (2′-OME) and 2′-O-Methoxyethyl (2′-MOE) ASOs. ASOs with this type of modification are incapable of activating RNAse H. Therefore, to induce RNAse H activation, chimeric ASOs have been developed in which a central gap region consisting of a phosphorothioate deoxyribose core is flanked with nuclease resistant arms such as 2′-OME or 2′-MOE that possess greater nuclease resistance. A “gapmer” is produced as a result, in which RNAse H can sit in the central gap and activate target specific mRNA degradation, while the arms prevent the ASO degradation. ASOs in this category possess higher affinity for mRNA, show better tissue uptake, and have increased resistance to nucleases, longer in vivo half life, and lesser toxicity, as compared to the modified ASOs of the first class.

A further class of ASOs known in the art and that may be utilized in the ASOs of the disclosure contain modifications of the furanose ring along with modifications of the phosphate linkage, the ribose moiety, or the nucleotides. These modifications were designed to improve the nuclease stability, target affinity and pharmacokinetic profiles of the ASOs. Common examples of third category of ASOs are Locked nucleic acid (LNA), Peptide nucleic acid (PNA) and Morpholino phosphoroamidates (MF) ASOs in this category are more stable in biological fluids because of their high resistance to degradation by nucleases and peptidases. They also exhibit a strong hybridization affinity with the mRNA. Further, PNAs recognize double stranded DNA, and are able to modulate gene expression or induce mutation by strand invasion of chromosomal duplex DNA. ASOs in this category also do not activate RNAse H and rely on sterically hindering the ribosomal machinery to cause translational arrest. They do not bind to serum proteins as they are uncharged. Lack of charge reduces the odds of non-specific interactions but increases the rate of clearance from the body. Their electrostatically neutral backbones may reduce solubility and make uptake more difficult.

A representative list of preferred modified sugars includes but is not limited to bicyclic modified sugars (BNA's), including methyleneoxy (4′-CH2-O-2′) BNA and ethyleneoxy (4′-(CH2)2-O-2′ bridge) BNA; substituted sugars, especially 2′-substituted sugars having a 2′-F, 2′-OCH3 or a 2′-O(CH2)2-OCH3 substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

c. Modified Internucleotide Linkages

Nucleic acid therapeutics may further comprise at least one phosphorothioate or methylphosphonate internucleotide linkage. The phosphorothioate or methylphosphonate internucleotide linkage modification may occur on any nucleotide of the sense strand or antisense strand or both (in nucleic acid therapeutics including a sense strand) in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In certain embodiments, the ASOs of the disclosure comprise one or more nucleoside subunits connected by phosphorus linkages including phosphodiester, phosphorothioate, 3 ‘(or -5’)deoxy-3 ‘-(or -5’)thio-phosphorothioate, phosphorodithioate, phosphoroselenates, 3′-(or -5′)deoxy phosphinates, borano phosphates, 3′-(or 5′-)amino phosphoramidates, hydrogen phosphonates, borano phosphate esters, phosphoramidates, alkyl or aryl phosphonates and phosphotriester phosphorus linkages. In some embodiments, the ASOs of the disclosure comprise nucleoside subunits connected by carbonate, carbamate, silyl, sulfur, sulfonate, sulfonamide, formacetal, thioformacetyl, oxime, methyleneimino, methylenemethylimino, methylenehydrazo, methylenedimethylhydrazo and methyleneoxymethylimino linkages.

For example, one class of modified ASO described in the art and that may be utilized in the ASOs of the disclosure are those that have one of the non-bridging oxygen atoms in the phosphate group of the ASO replaced with either a sulfur group (phosphorothioates), a methyl group (methyl phosphonates) or an amine group (phosphoramidates). These ASOs have greater resistance to nucleases and longer plasma half life as compared with phosphodiester oligonucleotides. They are capable of activating RNAse H, carry negative charges which facilitate their delivery to cells, and have suitable pharmacokinetics. Among these modifications, phosphorothioate modifications are used most widely. For example, Vitravene, an FDA approved ASO drug, and most of the other ASO drugs in clinical trials are phosphorothioate ASOs.

In addition, the bases in nucleotide may be joined by a linkage other than a phosphodiester bond, so long as it does not interfere with hybridization. Thus, inhibitory nucleic acids may be peptide nucleic acids in which the constituent bases are joined by peptide bonds rather than phosphodiester linkages. The inhibitory nucleic acids may be prepared by converting the RNA to cDNA using known methods (see, e.g., Ausubel et. al., Current Protocols in Molecular Biology Wiley 1999). The inhibitory nucleic acids can also be cRNA (see, e.g., Park et. al., (2004) Biochem. Biophys. Res. Commun. 325(4):1346-52).

B. Small Interfering RNAs

siRNA (e.g., siNA) are well known in the art. For example, siRNA and double-stranded RNA have been described in U.S. Pat. Nos. 6,506,559 and 6,573,099, as well as in U.S. Patent Applications 2003/0051263, 2003/0055020, 2004/0265839, 2002/0168707, 2003/0159161, and 2004/0064842, all of which are herein incorporated by reference in their entirety.

Within a siRNA, the components of a nucleic acid need not be of the same type or homogenous throughout (e.g., a siRNA may comprise a nucleotide and a nucleic acid or nucleotide analog). Typically, siRNA form a double-stranded structure; the double-stranded structure may result from two separate nucleic acids that are partially or completely complementary. In certain embodiments of the present disclosure, the siRNA may comprise only a single nucleic acid (polynucleotide) or nucleic acid analog and form a double-stranded structure by complementing with itself (e.g., forming a hairpin loop). The double-stranded structure of the siRNA may comprise, comprise at least, or comprise at most 16, 20, 25, 30, 35, 40, 45, 50, 60, 65, 70, 75, 80, 85, 90, 100, 150, 200, 250, 300, 350, 400, 450, 500 or more contiguous nucleobases, including all ranges and values therein. The siRNA may comprise 17 to 35 contiguous nucleobases, 18 to 30 contiguous nucleobases, 19 to 25 nucleobases, 20 to 23 contiguous nucleobases, 20 to 22 contiguous nucleobases, or 21 contiguous nucleobases that hybridize with a complementary nucleic acid (which may be another part of the same nucleic acid or a separate complementary nucleic acid) to form a double-stranded structure.

Agents of the present disclosure useful for practicing the methods of the present disclosure include, but are not limited to siRNAs. Typically, introduction of double-stranded RNA (dsRNA), which may alternatively be referred to herein as small interfering RNA (siRNA), induces potent and specific gene silencing, a phenomenon called RNA interference or RNAi. RNA interference has been referred to as “cosuppression,” “post-transcriptional gene silencing,” “sense suppression,” and “quelling.” RNAi is an attractive biotechnological tool because it provides a means for knocking out the activity of specific genes.

In designing RNAi there are several factors that need to be considered, such as the nature of the siRNA, the durability of the silencing effect, and the choice of delivery system. To produce an RNAi effect, the siRNA that is introduced into the organism will typically contain exonic sequences. Furthermore, the RNAi process is homology dependent, so the sequences must be carefully selected so as to maximize gene specificity, while minimizing the possibility of cross-interference between homologous, but not gene-specific sequences. Preferably the siRNA exhibits or exhibits greater than 80%, 85%, 90%, 95%, 98%, or even 100% identity, or any range or value derivable therein, between the sequence of the siRNA and the gene to be inhibited. Sequences less than about 80% identical to the target gene are substantially less effective. Thus, the greater homology between the siRNA and the gene to be inhibited, the less likely expression of unrelated genes will be affected.

In addition, the size of the siRNA is an important consideration. In some embodiments, the present disclosure relates to siRNA molecules that include, include at least, or include at most 19-25 nucleotides, or any range or value derivable therein, and are able to modulate gene expression. In the context of the present disclosure, the siRNA is, in some embodiments, less than 500, 200, 100, 50, or 25 nucleotides in length. In some embodiments, the siRNA is from about 19 nucleotides to about 25 nucleotides in length.

A target gene generally means a polynucleotide comprising a region that encodes a polypeptide, or a polynucleotide region that regulates replication, transcription, or translation or other processes important to expression of the polypeptide, or a polynucleotide comprising both a region that encodes a polypeptide and a region operably linked thereto that regulates expression. Any gene being expressed in a cell can be targeted. Preferably, a target gene is one involved in or associated with the progression of cellular activities important to disease or of particular interest as a research object.

siRNA can be obtained from commercial sources, natural sources, or can be synthesized using any of a number of techniques well-known to those of ordinary skill in the art. For example, one commercial source of predesigned siRNA is Ambion®, Austin, Tex. Another is Qiagen® (Valencia, Calif.). An inhibitory nucleic acid that can be applied in the compositions and methods of the present disclosure may be any nucleic acid sequence that has been found by any source to be a validated downregulator of a protein of interest. Without undue experimentation and using the disclosure of this disclosure, it is understood that additional siRNAs can be designed and used to practice the methods of the disclosure.

The siRNA may also comprise an alteration of one or more nucleotides. Such alterations can include the addition of non-nucleotide material, such as to the end(s) of the 19 to 25 nucleotide RNA or internally (at one or more nucleotides of the RNA). In certain aspects, the RNA molecule contains a 3′-hydroxyl group. Nucleotides in the RNA molecules of the present disclosure can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incorporation) can be found in U.S. Application Publication 2004/0019001 and U.S. Pat. No. 6,673,611 (each of which is incorporated by reference in its entirety). Collectively, all such altered nucleic acids or RNAs described above are referred to as modified siRNAs.

As exosomes are known to comprise DICER and active RNA processing RISC complex (see PCT Publn. WO 2014/152622, which is incorporated herein by reference in its entirety), shRNA transfected into exosomes can mature into RISC-complex bound siRNA within the exosomes themselves. Alternatively, mature siRNA can itself be transfected into exosomes or liposomes. Any inhibitory nucleic acid can be applied in the compositions and methods of the present disclosure if such inhibitory nucleic acid has been found by any source to be a validated downregulator of a protein of interest.

### III. TREATMENT OF DISEASES

A number of serious diseases of mammals, including humans, are associated with fibrosis. As used herein, “fibrosis” includes any condition involving the formation of fibrous tissue (whether or not such formation is desirable or undesirable). Such conditions include, but are not limited to: connective tissue inflammation, fibroma formation (fibromatosis), fibrosis (including lung fibrosis and liver fibrosis), fibroelastosis (endocardial fibers), fibromyopathy formation, fibroid formation, fibroidoma formation, fibromyxoma formation, and fibrocystitis (including cystic fibrosis).

In some embodiments, fibrosis that can be treated in an animal using an inhibitor of STAT3 expression include, but are not limited to liver fibrosis, lung fibrosis, pulmonary fibrosis, cystic fibrosis, idiopathic pulmonary fibrosis (IPF), or radiation-induced lung injury. In some embodiments, fibrosis that can be treated include, but are not limited to, lung fibrosis (e.g., pulmonary fibrosis, cystic fibrosis, idiopathic pulmonary fibrosis (IPF), radiation-induced lung injury, or radiation-induced lung injury resulting from treatment for cancer), skin fibrosis, kidney fibrosis, liver fibrosis (e.g., cirrhosis), gastrointestinal fibrosis (e.g., fibrosis of the gastrointestinal tract, fibrosis associated with gastrointestinal inflammation, fibrosis associated with inflammatory bowel disease, fibrosis associated with ulcerative colitis, fibrosis associated with Crohn's disease, intestine fibrosis, small intestine fibrosis, ilium fibrosis, cecum fibrosis, or colon fibrosis), heart fibrosis (e.g., atrial fibrosis, endomyocardial fibrosis, or myocardial infarction), brain fibrosis (e.g., glial scar), or other forms of fibrosis including but not limited to arterial stiffness, arthrofibrosis (e.g., knee, shoulder, or other joints), Crohn's disease (e.g., intestine), dupuytren's contracture (e.g., hand or finger), keloid (e.g., skin), mediastinal fibrosis (e.g., soft tissue of the mediastinum), myelofibrosis (e.g., bone marrow), peyronie's disease (e.g., penis), nephrogenic systemic fibrosis (e.g., skin), progressive massive fibrosis (e.g., a complication of coal workers' pneumoconiosis), retroperitoneal fibrosis (e.g., soft tissue of the retroperitoneum), scleroderma/systemic sclerosis (e.g., skin or lung), adhesive capsulitis (e.g., shoulder), or other organ fibrosis.

Animals that can be treated include but are not limited to mammals, rodents, primates, monkeys (e.g., macaque, rhesus macaque, pig tail macaque), humans, canine, feline, porcine, avian (e.g., chicken), bovine, mice, rabbits, and rats. As used herein, the term “individual,” “subject,” and “patient” are used interchangeably and can refer to both human and non-human subjects. In some instances, the animal is in need of the treatment (e.g., by showing signs of disease or fibrosis).

As used herein, the term “treating” (and its variations, such as “treatment”) is to be considered in its broadest context. In particular, the term “treating” does not necessarily imply that an animal is treated until total recovery. Accordingly, “treating” includes amelioration of the symptoms, relief from the symptoms or effects associated with a condition, decrease in severity of a condition, or preventing, preventively ameliorating symptoms, or otherwise reducing the risk of developing a particular condition. As used herein, reference to “treating” an animal includes but is not limited to prophylactic treatment and therapeutic treatment. Any of the compositions (e.g., pharmaceutical compositions) described herein can be used to treat an animal.

As related to treating fibrosis (e.g., liver fibrosis, lung fibrosis, pulmonary fibrosis, cystic fibrosis, idiopathic pulmonary fibrosis (IPF), or radiation-induced lung injury), treating can include but is not limited to prophylactic treatment and therapeutic treatment. As such, treatment can include, but is not limited to: preventing fibrosis (e.g., liver fibrosis, lung fibrosis, pulmonary fibrosis, cystic fibrosis, idiopathic pulmonary fibrosis (IPF), or radiation-induced lung injury); reducing the risk of fibrosis (e.g., liver fibrosis, lung fibrosis, pulmonary fibrosis, cystic fibrosis, idiopathic pulmonary fibrosis (IPF), or radiation-induced lung injury); ameliorating or relieving symptoms of fibrosis (e.g., liver fibrosis, lung fibrosis, pulmonary fibrosis, cystic fibrosis, idiopathic pulmonary fibrosis (IPF), or radiation-induced lung injury); eliciting a bodily response against fibrosis (e.g., liver fibrosis, lung fibrosis, pulmonary fibrosis, cystic fibrosis, idiopathic pulmonary fibrosis (IPF), or radiation-induced lung injury); inhibiting the development or progression of fibrosis (e.g., liver fibrosis, lung fibrosis, pulmonary fibrosis, cystic fibrosis, idiopathic pulmonary fibrosis (IPF), or radiation-induced lung injury); inhibiting or preventing the onset of symptoms associated with fibrosis (e.g., liver fibrosis, lung fibrosis, pulmonary fibrosis, cystic fibrosis, idiopathic pulmonary fibrosis (IPF), or radiation-induced lung injury); reducing the severity of fibrosis (e.g., liver fibrosis, lung fibrosis, pulmonary fibrosis, cystic fibrosis, idiopathic pulmonary fibrosis (IPF), or radiation-induced lung injury); causing a regression of fibrosis (e.g., liver fibrosis, lung fibrosis, pulmonary fibrosis, cystic fibrosis, idiopathic pulmonary fibrosis (IPF), or radiation-induced lung injury) or one or more of the symptoms associated with fibrosis (e.g., a decrease in the amount of fibrosis); causing remission of fibrosis (e.g., liver fibrosis, lung fibrosis, pulmonary fibrosis, cystic fibrosis, idiopathic pulmonary fibrosis (IPF), or radiation-induced lung injury); or preventing relapse of fibrosis (e.g., liver fibrosis, lung fibrosis, pulmonary fibrosis, cystic fibrosis, idiopathic pulmonary fibrosis (IPF), or radiation-induced lung injury). In some embodiments, treating does not include prophylactic treatment of fibrosis (e.g., preventing or ameliorating future fibrosis).

Treatment of an animal (e.g., human) can occur using any suitable administration method (such as those disclosed herein) and using any suitable amount of a STAT3 expression inhibitor (e.g., siRNA or ASO). In some embodiments, methods of treatment comprise treating an animal for fibrosis (e.g., liver fibrosis, lung fibrosis, pulmonary fibrosis, cystic fibrosis, idiopathic pulmonary fibrosis (IPF), or radiation-induced lung injury). Some embodiments of the disclosure include a method for treating a subject (e.g., an animal such as a human or primate) with a composition comprising one or more STAT3 expression inhibitors (e.g., siRNA or ASO) (e.g., a pharmaceutical composition) which comprises one or more administrations of one or more such compositions; the compositions may be the same or different if there is more than one administration.

Treatment using a STAT3 expression inhibitor may result in decreased expression of one or more genes associated with (e.g., regulated by) STAT3 activity. In some embodiments, a treatment of the disclosure reduces expression of one or more genes associated with STAT3 activity including, for example, Col1a1, Acta2, Col1a2, and/or Vim. In some embodiments, a treatment of the disclosure reduces expression of Col1a1 in target cells (e.g., hepatic cells) of the patient. Example target cells of the disclosure include hepatic cells, such as activated hepatic stellate cells and αSMA⁺ myofibroblasts.

In some embodiments, other fibrosis treatments are optionally included, and can be used with the inventive treatments described herein. Other fibrosis treatments can include any known fibrosis treatment that is suitable to treat fibrosis. Examples of known fibrosis treatments include but are not limited to administration of: antibiotics (e.g., penicillins, methicillin, oxacillin, nafcillin, cabenicillin, ticarcillin, piperacillin, mezlocillin, azlocillin, ticarcillin clavulanic acid, piperacillin tazobactam, cephalosporins, cephalexin, cefdinir, cefprozil, cefaclor, cefepime, sulfa, sulfamethoxazole, trimethoprim, erythromycin/sulfisoxazole, macrolides, erythromycin, clarithromycin, azithromycin, tetracyclines, tetracycline, doxycycline, minocycline, tigecycline, vancomycin, imipenem, meripenem, colistimethate/colistin, aminoglycosides, tobramycin, amikacin, gentamicin, quinolones, aztreonam, or linezolid), anti-inflammatory drugs (e.g., NSAIDs, aspirin, ibuprofen, naproxen, corticosteroids, cortisol, corticosterone, cortisone, or aldosterone), bronchodilators (e.g., albuterol or levalbuterol hydrochloride), mucus thinners (e.g., hypertonic saline or Domase alfa), and antifibrotic medications (e.g., pirfenidone, nintedanib, N-acetylcysteine, ivacaftor, or lumacaftor/ivacaftor). Other fibrosis treatment can also include administering a non-drug respiratory therapy such as but not limited to airway clearance techniques (e.g., postural drainage and chest percussion, exercise, breathing exercises, or use of mechanical equipment such as high-frequency chest compression vest or positive expiratory pressure therapy). Other fibrosis treatment can also include organ transplantation (e.g., lung, skin, kidney, liver, heart, small intestine, or colon). It is contemplated that one or more other fibrosis treatments may be excluded in embodiments of the disclosure.

In some embodiments, administration of an opioid receptor inhibitor, naltrexone, pirfenidone, nintedanib, or a combination thereof can be used as part of the treatment regime (i.e., as another fibrosis treatment); administration of an opioid receptor inhibitor, naltrexone, pirfenidone, nintedanib, or a combination thereof, can include separate administrations (i.e., in a separate composition from the STAT3 expression inhibitor) or can be added to the composition comprising the STAT3 expression inhibitor.

In some embodiments, additional optional treatments (e.g., as another fibrosis treatment) can also include one or more of surgical intervention, hormone therapies, immunotherapy, and adjuvant systematic therapies. It is contemplated that one or more additional optional treatments may be excluded in embodiments of the disclosure.

For the treatment of disease, the appropriate dosage of a therapeutic composition will depend on the type of disease to be treated, as defined above, the severity and course of the disease, the patient's clinical history and response to the agent, and the discretion of the attending physician. The agent is suitably administered to the patient at one time or over a series of treatments.

Therapeutic and prophylactic methods and compositions can be provided in a combined amount effective to achieve the desired effect. A tissue, tumor, or cell can be contacted with one or more compositions or pharmacological formulation(s) comprising one or more of the agents, or by contacting the tissue, tumor, and/or cell with two or more distinct compositions or formulations. Also, it is contemplated that such a combination therapy can be used in conjunction with chemotherapy, radiotherapy, surgical therapy, or immunotherapy.

Administration in combination can include simultaneous administration of two or more agents in the same dosage form, simultaneous administration in separate dosage forms, and separate administration. That is, the subject therapeutic composition and another therapeutic agent can be formulated together in the same dosage form and administered simultaneously. Alternatively, subject therapeutic composition and another therapeutic agent can be simultaneously administered, wherein both the agents are present in separate formulations. In another alternative, the therapeutic agent can be administered just followed by the other therapeutic agent or vice versa. In the separate administration protocol, the subject therapeutic composition and another therapeutic agent may be administered a few minutes apart, or a few hours apart, or a few days apart.

### IV. PHARMACEUTICAL COMPOSITIONS

It is contemplated that exosomes that express or comprise a therapeutic agent can be administered systemically or locally to enhance telomerase activity. They can be administered intravenously, intrathecally, and/or intraperitoneally. They can be administered alone or in combination with a second drug.

It is not intended that the present invention be limited by the particular nature of the therapeutic preparation. For example, such compositions can be provided in formulations together with physiologically tolerable liquid, gel, solid carriers, diluents, or excipients. These therapeutic preparations can be administered to mammals for veterinary use, such as with domestic animals, and clinical use in humans in a manner similar to other therapeutic agents. In general, the dosage required for therapeutic efficacy will vary according to the type of use and mode of administration, as well as the particular requirements of individual subjects.

Where clinical applications are contemplated, it may be necessary to prepare pharmaceutical compositions comprising exosomes in a form appropriate for the intended application. Generally, pharmaceutical compositions may comprise an effective amount of one or more exosomes and/or additional agents dissolved or dispersed in a pharmaceutically acceptable carrier. The phrases “pharmaceutical or pharmacologically acceptable” refers to molecular entities and compositions that do not produce an adverse, allergic, or other untoward reaction when administered to an animal, such as, for example, a human, as appropriate. The preparation of a pharmaceutical composition comprising exosomes as disclosed herein, or additional active ingredient will be known to those of skill in the art in light of the present disclosure, as exemplified by Remington's Pharmaceutical Sciences, 18th Ed., 1990, incorporated herein by reference. Moreover, for animal (e.g., human) administration, it will be understood that preparations should meet sterility, pyrogenicity, general safety, and purity standards as required by the FDA Office of Biological Standards.

Further in accordance with certain aspects of the present disclosure, the composition suitable for administration may be provided in a pharmaceutically acceptable carrier with or without an inert diluent. As used herein, “pharmaceutically acceptable carrier” includes any and all aqueous solvents (e.g., water, alcoholic/aqueous solutions, ethanol, saline solutions, parenteral vehicles, such as sodium chloride, Ringer's dextrose, etc.), non-aqueous solvents (e.g., fats, oils, polyol (for example, glycerol, propylene glycol, and liquid polyethylene glycol, and the like), vegetable oil, and injectable organic esters, such as ethyloleate), lipids, liposomes, dispersion media, coatings (e.g., lecithin), surfactants, antioxidants, preservatives (e.g., antibacterial or antifungal agents, anti-oxidants, chelating agents, inert gases, parabens (e.g., methylparabens, propylparabens), chlorobutanol, phenol, sorbic acid, thimerosal or combinations thereof), isotonic agents (e.g., sugars and sodium chloride), absorption delaying agents (e.g., aluminum monostearate and gelatin), salts, drugs, drug stabilizers, gels, resins, fillers, binders, excipients, disintegration agents, lubricants, sweetening agents, flavoring agents, dyes, fluid and nutrient replenishers, such like materials and combinations thereof, as would be known to one of ordinary skill in the art. The carrier should be assimilable and includes liquid, semi-solid, i.e., pastes, or solid carriers. In addition, if desired, the compositions may contain minor amounts of auxiliary substances, such as wetting or emulsifying agents, stabilizing agents, or pH buffering agents. The pH and exact concentration of the various components in a pharmaceutical composition are adjusted according to well-known parameters. The proper fluidity can be maintained, for example, by the use of a coating, such as lecithin, by the maintenance of the required particle size in the case of dispersion, and by the use of surfactants.

A pharmaceutically acceptable carrier is particularly formulated for administration to a human, although in certain embodiments it may be desirable to use a pharmaceutically acceptable carrier that is formulated for administration to a non-human animal but that would not be acceptable (e.g., due to governmental regulations) for administration to a human. Except insofar as any conventional carrier is incompatible with the active ingredient (e.g., detrimental to the recipient or to the therapeutic effectiveness of a composition contained therein), its use in the therapeutic or pharmaceutical compositions is contemplated. In accordance with certain aspects of the present disclosure, the composition is combined with the carrier in any convenient and practical manner, i.e., by solution, suspension, emulsification, admixture, encapsulation, absorption, and the like. Such procedures are routine for those skilled in the art.

Certain embodiments of the present disclosure may comprise different types of carriers depending on whether it is to be administered in solid, liquid, or aerosol form, and whether it needs to be sterile for the route of administration, such as injection. The compositions can be administered intravenously, intradermally, transdermally, intrathecally, intraarterially, intraperitoneally, intranasally, intravaginally, intrarectally, intramuscularly, subcutaneously, mucosally, orally, topically, locally, by inhalation (e.g., aerosol inhalation), by injection, by infusion, by continuous infusion, by localized perfusion bathing target cells directly, via a catheter, via a lavage, in lipid compositions (e.g., liposomes), or by other methods or any combination of the forgoing as would be known to one of ordinary skill in the art (see, for example, Remington's Pharmaceutical Sciences, 18th Ed., 1990, incorporated herein by reference).

The exosomes can be formulated for parenteral administration, e.g., formulated for injection via the intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

Upon formulation, solutions will be administered in a manner compatible with the dosage formulation and in such amount as is therapeutically effective. The formulations are easily administered in a variety of dosage forms, such as formulated for parenteral administrations, such as injectable solutions, or aerosols for delivery to the lungs, or formulated for alimentary administrations, such as drug release capsules and the like.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present disclosure administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. As another example, a dose may also comprise from about 1 billion to about 500 billion exosomes (this such range includes intervening doses) or more per administration, and any range derivable therein. In non-limiting examples of a derivable range from the numbers listed herein, a range of about 1 million exosomes to about 500 billion exosomes, about 5 million exosomes to about 250 billion exosomes, etc., can be administered. In one example, a dose may comprise about 150 billion exosomes in a 5 mL volume, and such dose may be administered to a human patient weighing 70 kg. The practitioner responsible for administration will, in any event, determine the concentration of active ingredient(s) in a composition and appropriate dose(s) for the individual subject.

The actual dosage amount of a composition administered to an animal patient can be determined by physical and physiological factors, such as body weight, severity of condition, the type of disease being treated, previous or concurrent therapeutic interventions, idiopathy of the patient, and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least or at most about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared in such a way that a suitable dosage will be obtained in any given unit dose of the compound. Factors, such as solubility, bioavailability, biological half-life, route of administration, product shelf life, as well as other pharmacological considerations, will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 milligram/kg/body weight or more per administration, and any range derivable therein. In non-limiting examples of a derivable range from the numbers listed herein, a range of about 5 milligram/kg/body weight to about 100 milligram/kg/body weight, about 5 microgram/kg/body weight to about 500 milligram/kg/body weight, etc., can be administered, based on the numbers described above.

### V. KITS AND DIAGNOSTICS

In various aspects of the disclosure, a kit is envisioned containing the necessary components to purify exosomes from a body fluid or tissue culture medium. In other aspects, a kit is envisioned containing the necessary components to isolate exosomes and transfect them with a therapeutic nucleic acid, therapeutic protein, or an inhibitory RNA. The kit may comprise one or more sealed vials containing any of such components. In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass. The kit may further include an instruction sheet that outlines the procedural steps of the methods set forth herein, and will follow substantially the same procedures as described herein or are known to those of ordinary skill. The instruction information may be in a computer readable media containing machine-readable instructions that, when executed using a computer, cause the display of a real or virtual procedure of purifying exosomes from a sample and transfecting the exosomes with a therapeutic cargo.

### VI. EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Localization of Exosomes to the Livers of a Mouse Model of Liver Fibrosis Induced by CC14

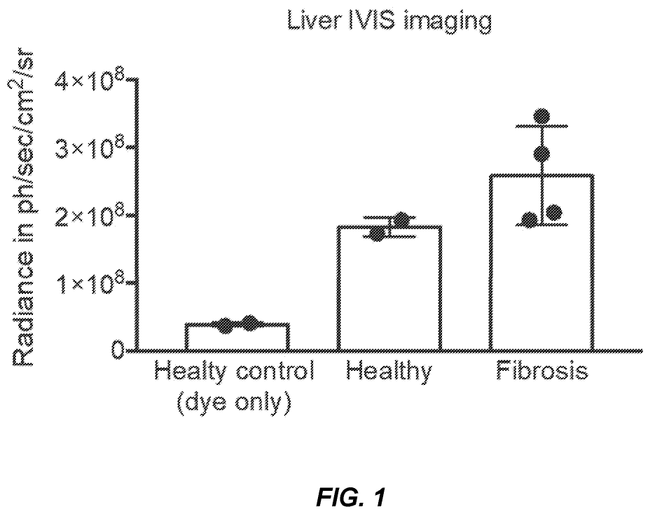

Liver fibrosis was induced in Balb/c adult mice with bi-weekly i.p. injections of CCl₄ for six weeks. Mice, both with and without liver fibrosis, were intravenously administered either 10⁹ dye-labeled exosomes or dye only. Three hours after administration, the mice were euthanized. Mice given DiR-labeled exosomes were assayed using IVIS imaging (FIG. 1). Mice given PHK67-labeled exosomes were assayed using confocal microscopy. Exosomes localized to the liver in both healthy and fibrotic mice; however, the level of localization was increased in the fibrotic mice relative to the healthy mice.

### Example 2—Reduction in Liver Fibrosis Following Administration of Exosomes Loaded with STAT3 Inhibitory RNA

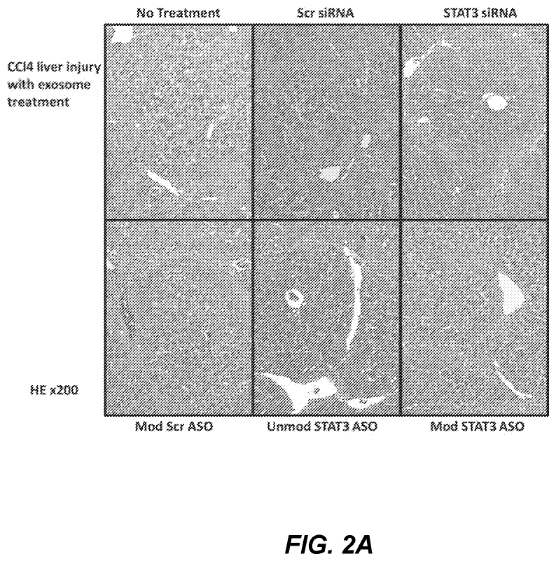

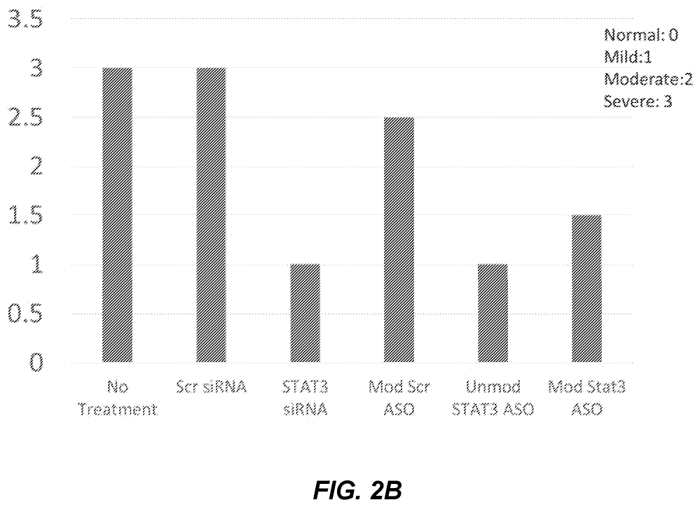

Liver fibrosis was induced in C57B16 adult mice with bi-weekly i.p. injections of CCl₄ for six weeks. Then MSC-derived exosomes (1.5 billion exosomes/dose comprising 1.5 μg siRNA or ASO/injection) were administered every 48 hours. The exosomes contained scrambled siRNA, STAT3 siRNA, unmodified STAT3 anti-sense oligonucleotide, modified scrambled anti-sense oligonucleotide, or modified STAT3 anti-sense oligonucleotide. Following treatment, the mice were euthanized, their livers were sectioned and processed for hematoxylin and eosin (H&E) staining, and the stained sections were imaged (FIG. 2A). The level of fibrosis was quantified (FIG. 2B). The results show that both STAT3 siRNA, unmodified STAT3 anti-sense oligonucleotide, and modified STAT3 anti-sense oligonucleotide reduced the level of fibrosis.

### Example 3—Reduction in Lung Fibrosis Following Administration of Exosomes Loaded with STAT3 Inhibitory RNA

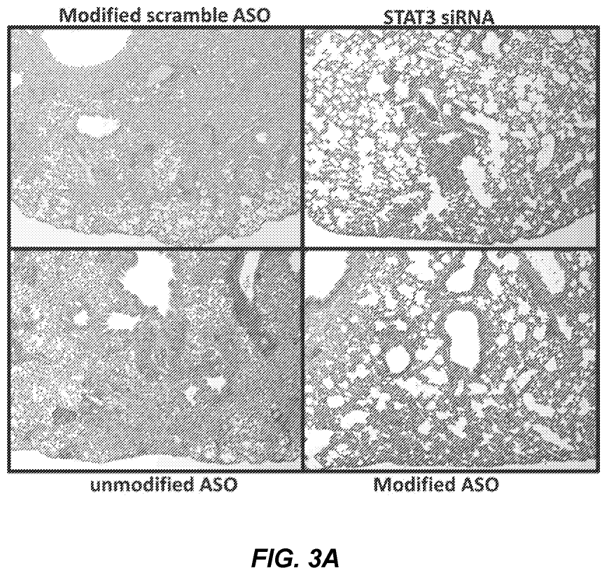

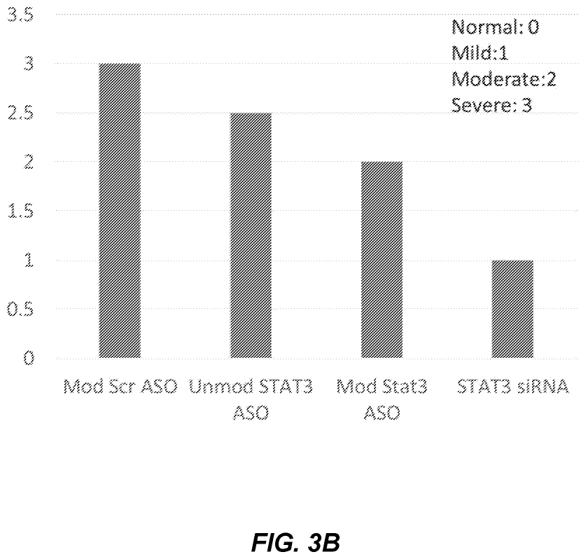

Lung fibrosis was induced in mice using bleomycin. Then MSC-derived exosomes (2 billion exosomes/dose comprising 5 μg siRNA or ASO/injection) were administered every 48 hours. The exosomes contained STAT3 siRNA, unmodified STAT3 anti-sense oligonucleotide, modified scrambled anti-sense oligonucleotide, or modified STAT3 anti-sense oligonucleotide. Following treatment, the mice were euthanized, their lungs were sectioned and processed for hematoxylin and eosin (H&E) staining, and the stained sections were imaged (FIG. 3A). The level of fibrosis was quantified (FIG. 3B). The results show that STAT3 siRNA provided the greatest decrease in the level of fibrosis.

### Example 4—Exosome-Mediated Therapeutic Targeting of STAT3 in Liver Fibrosis

Results

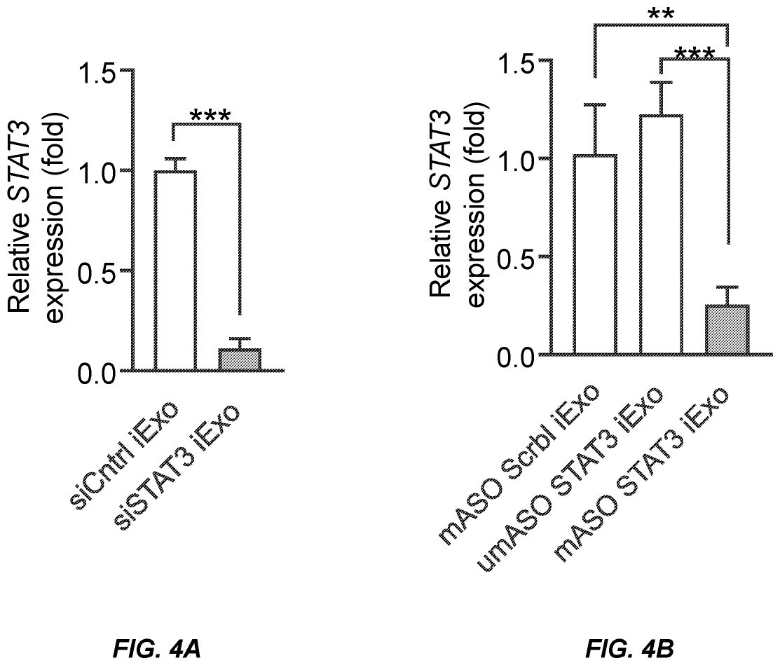

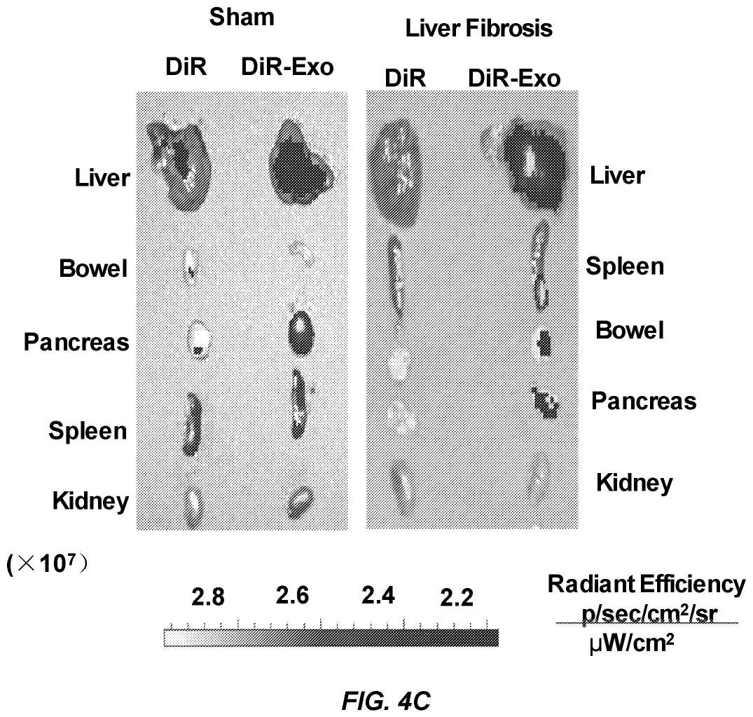

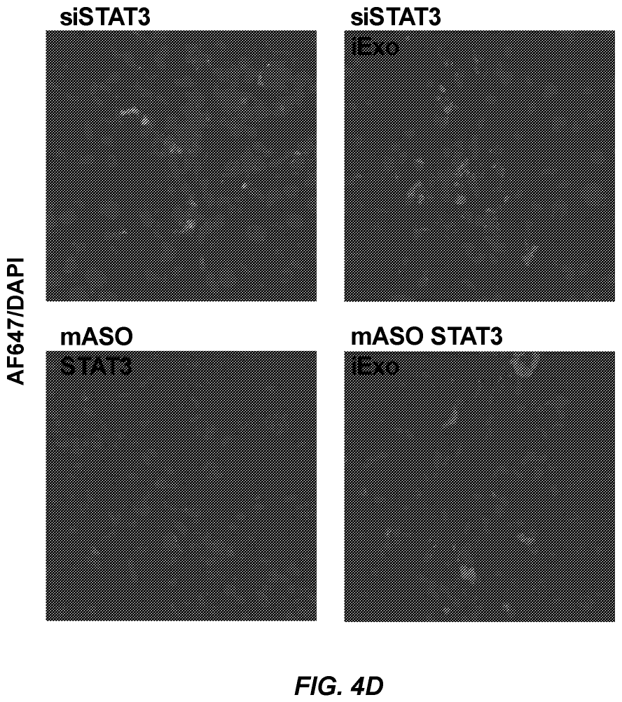

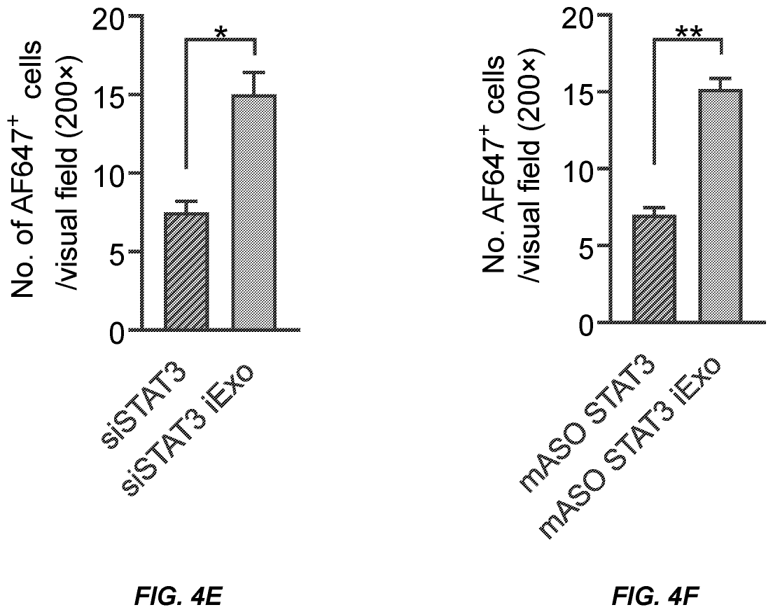

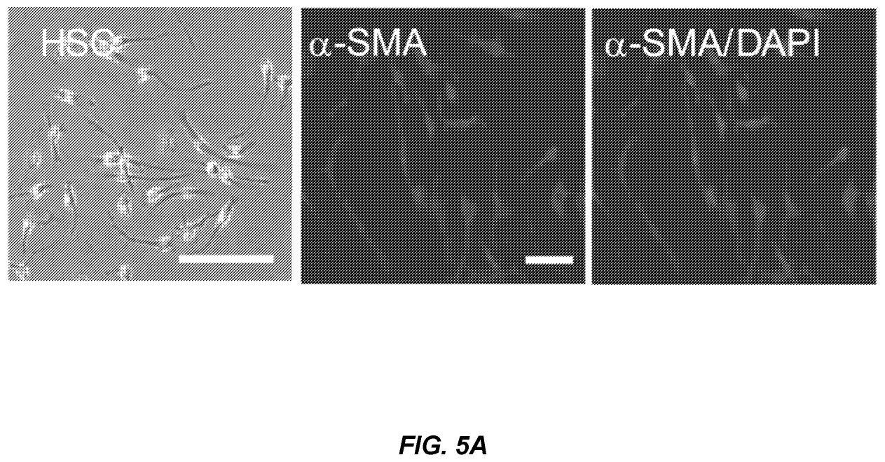

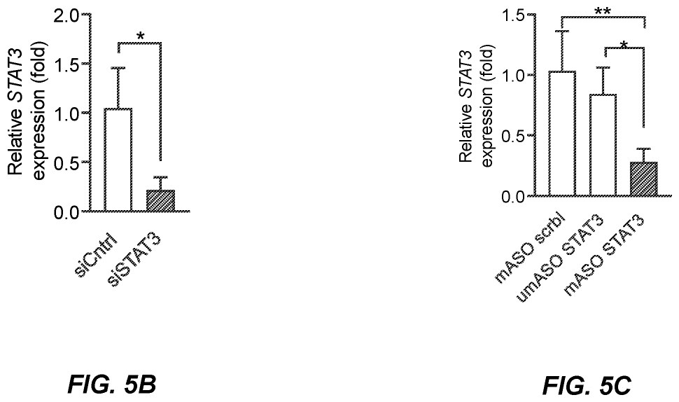

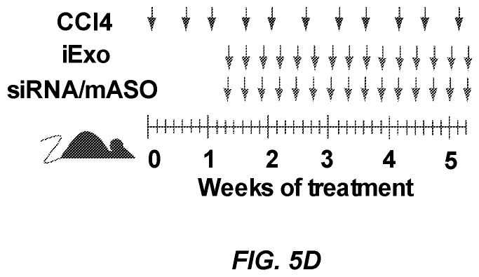

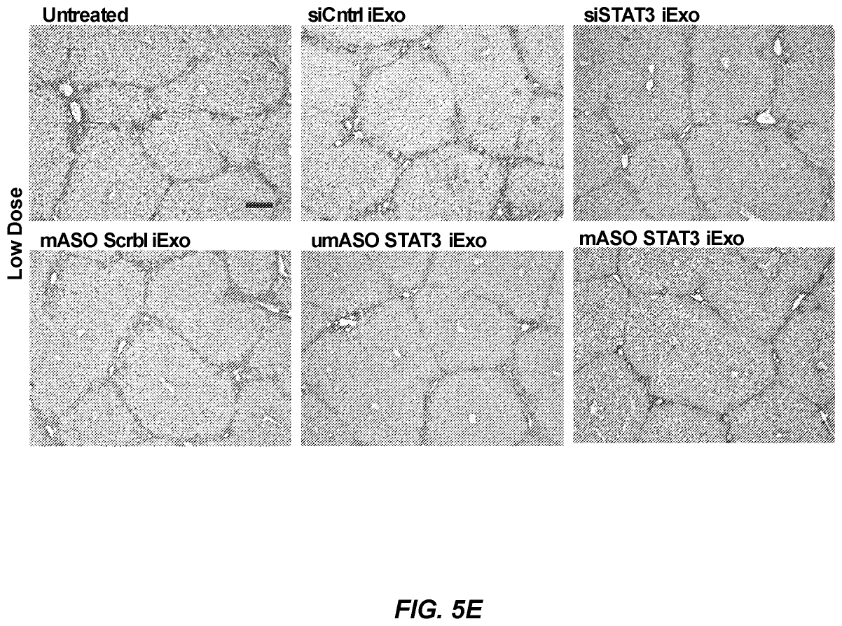

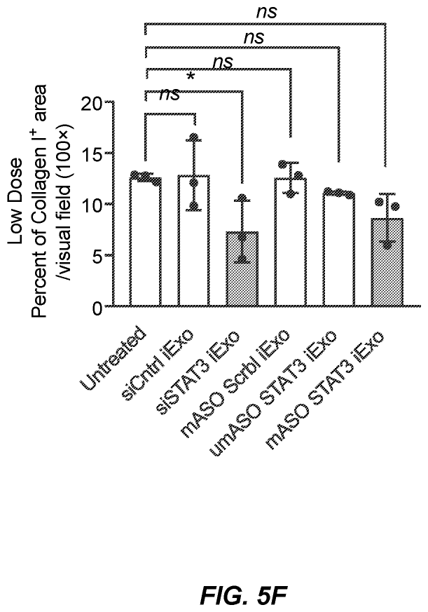

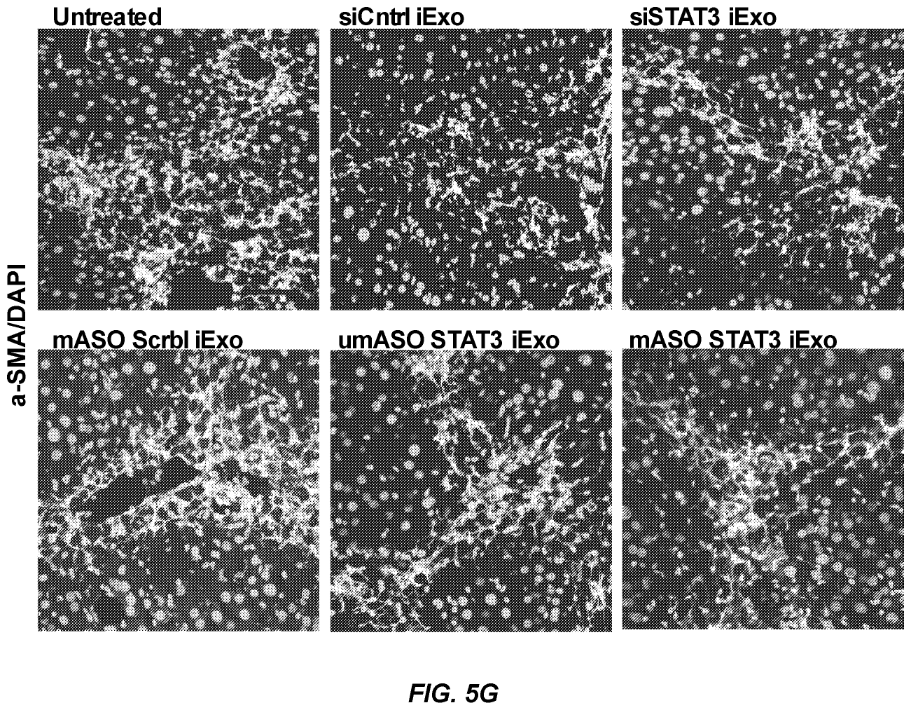

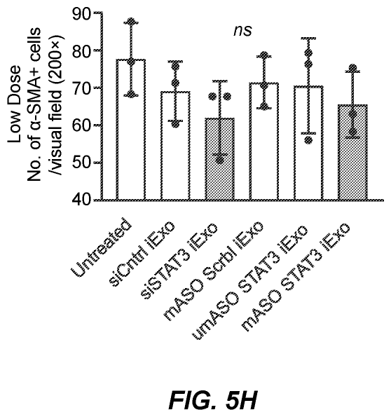

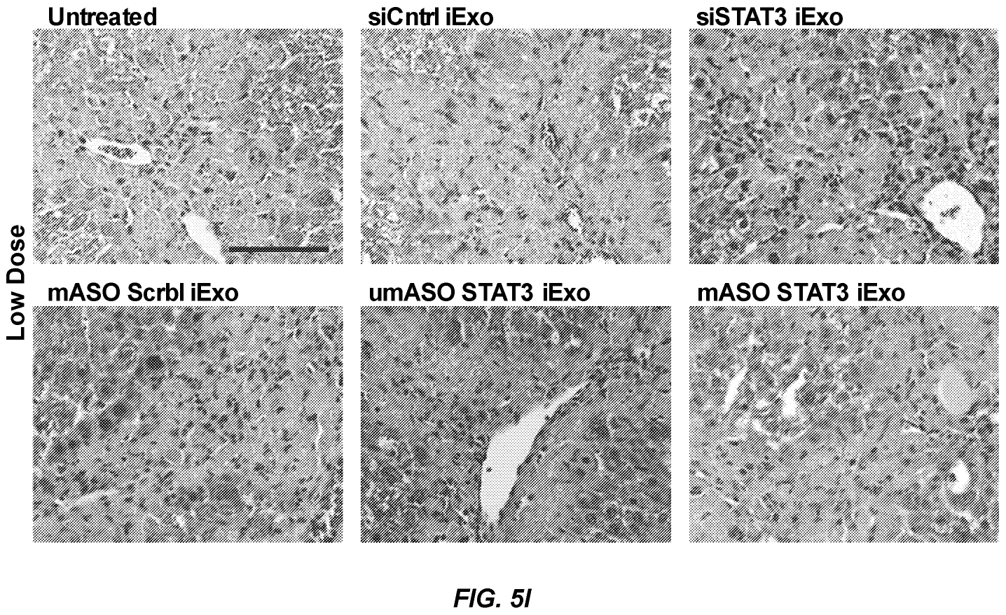

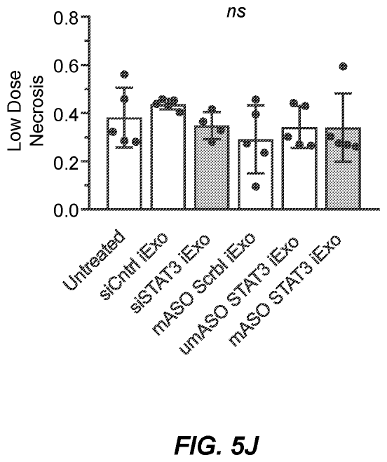

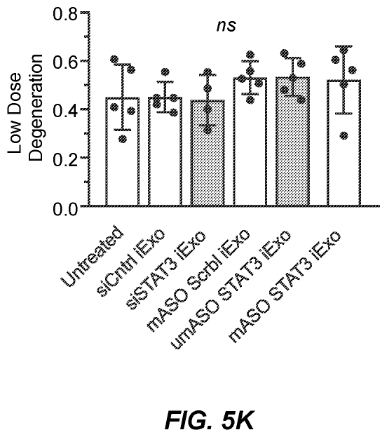

To determine the efficiency of iExosomes (exosomes engineered to deliver a nucleic acid payload) loaded with siRNA or ASO targeting STAT3, primary hepatic stellate cells (HSCs) isolated from wild-type (WT) mouse were cultured for 7 days, which led to the spontaneously activation of HSCs (FIG. 4A) (Zhai et al., 2019; Lu et al., 2014; Mederacke et al., 2013). The activation of HSCs is characterized by the expression of alpha-smooth muscle actin (α-SMA) (FIG. 4B). iExo^{siRNA-STAT3} or iExo^{mASO-STAT3} treatment significantly reduced Stat3 mRNA levels in HSCs (FIGS. 4C and 4D) with similar efficiency compared to lipid-based transfection reagent (FIGS. 5A and 5B).

The tropism of exogenously administered exosomes in mice was previously reported (Mendt et al., 2018; Kamerkar et al., 2017), which included several GI organs such as the pancreas and the liver. The inventors confirmed the liver tropism of human mesenchymal stromal cells (MSCs)-derived exosomes to the liver of healthy mice (FIG. 4C). To further investigate the biodistribution of exosomes in fibrotic tissue, DiR labeled exosomes were administered intraperitoneally (i.p.) into fibrotic liver and WT mice induced by carbon tetrachloride (CC14). The results showed that a specific accumulation signal associated with exosomes in the normal liver and pancreas, and lower amount of signal detected in the kidney, bowel and spleen (FIG. 4C). However, the fibrotic liver exhibited higher enrichment of DiR labeled exosomes compared to the healthy liver. Furthermore, the inventors electroporated Alexa-Fluor 647 (AF647)-tagged siSTAT3 or modified ASO (mASO) STAT3 into exosomes (iExo^{siRNA647-STAT3} or iExo^{mASO647-STAT3}), following i.p. injection (24 hours later) of tagged iExosomes/siSTAT3/mASO STAT3, and the results revealed accumulation of fluorescently labeled siRNA and ASO in the liver at a higher rate than naked siRNA or ASO (FIGS. 4D-F).

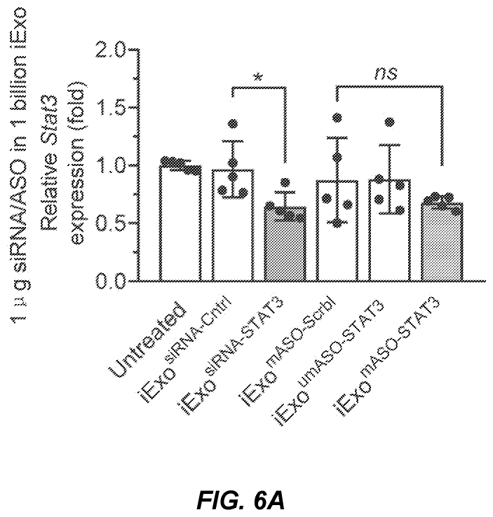

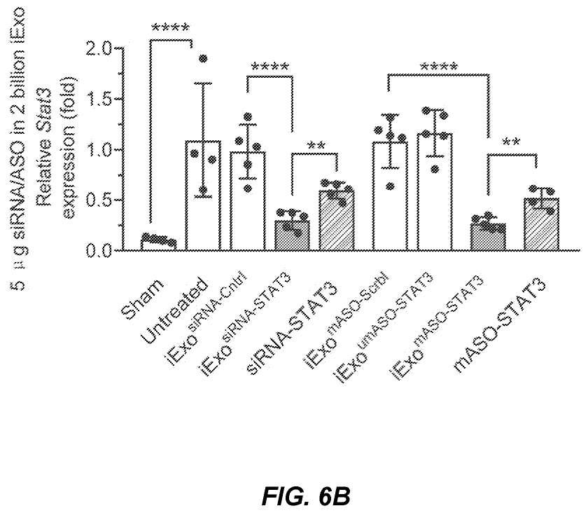

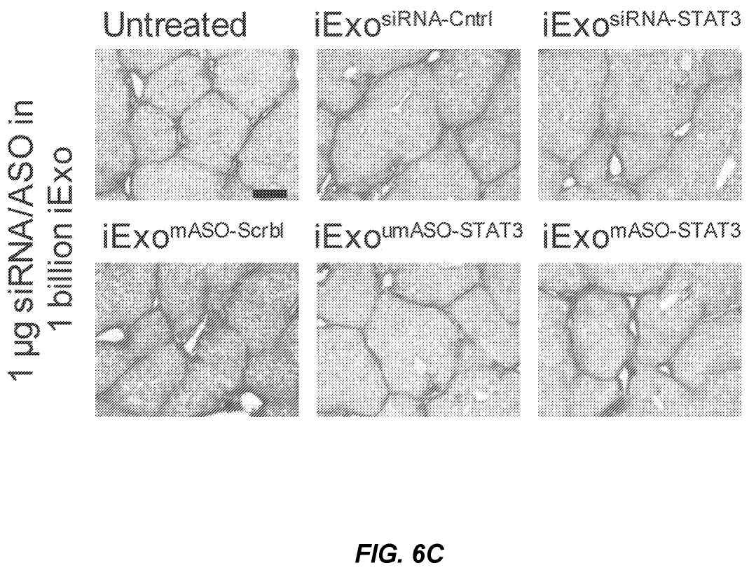

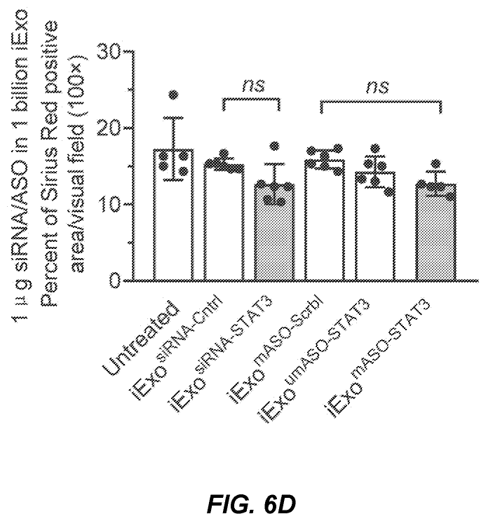

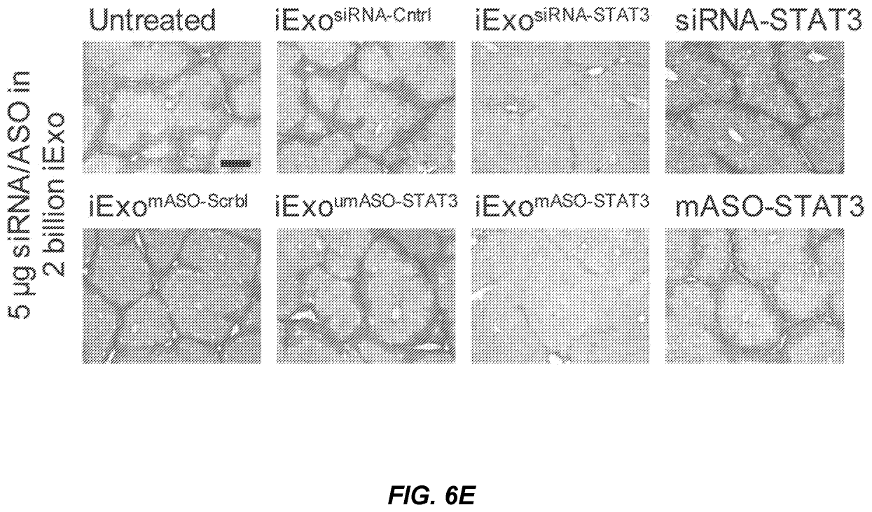

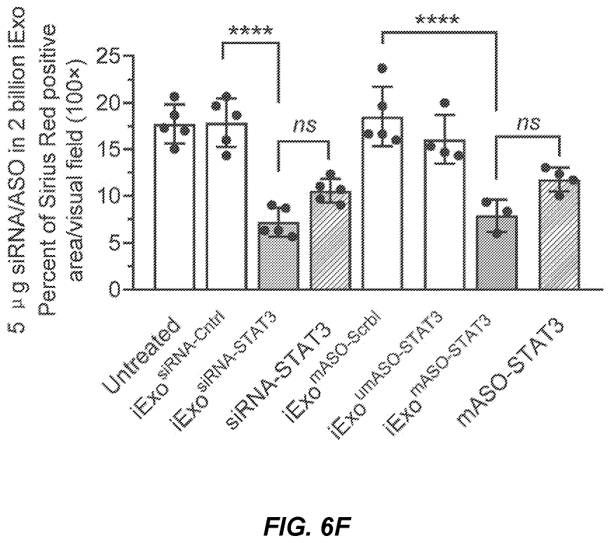

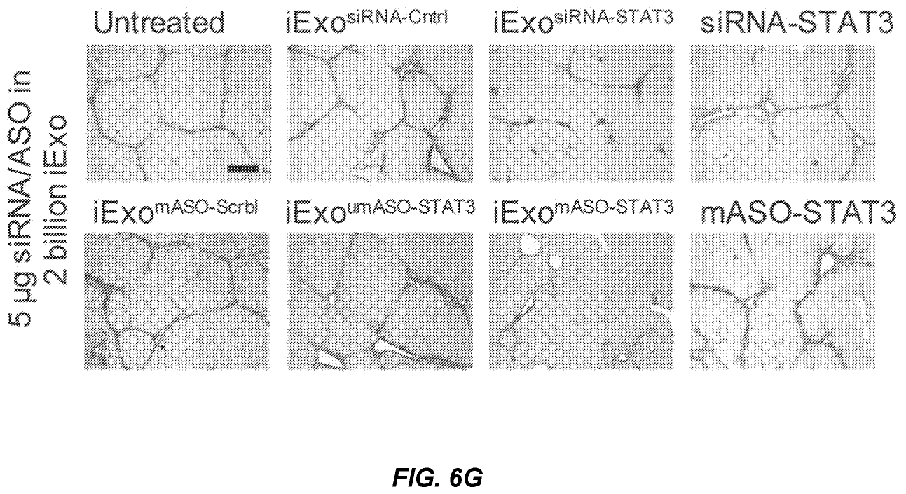

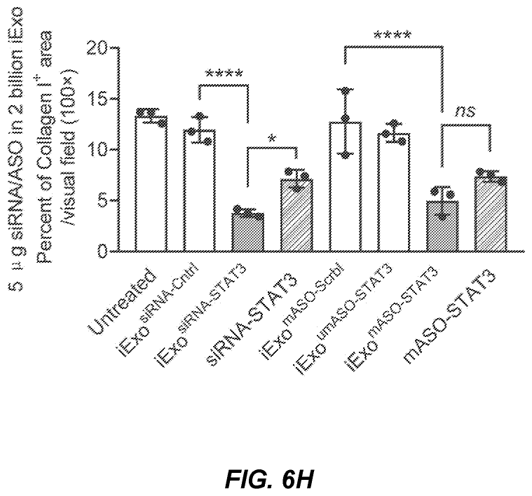

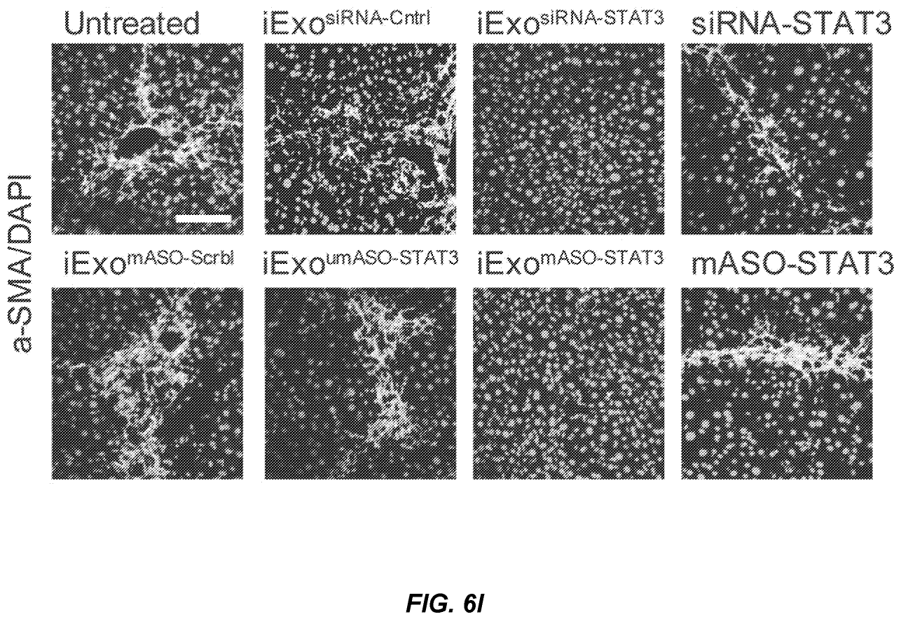

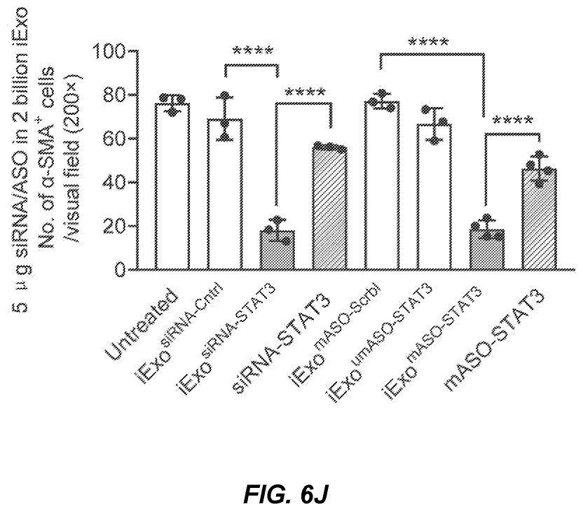

In order to verify the function of iExosomes in the treatment of liver fibrosis in vivo, WT mice were subjected to i.p. injection of CC14 twice weekly to induce chronic liver fibrosis (FIG. 5D). The mice were also treated with naked siRNA or ASO, or iExo^{siRNA} or iExo^{mASO} on day 9 post induction of fibrosis (FIG. 5D). iExosomes with siRNA or ASO targeting STAT3 were administered at two dosages, 1 billion exosomes electroporated with 1 μg siRNA or ASO (1 μg/1 billion iExo) and 2 billion exosomes electroporated with 5 μg siRNA or ASO (5 μg/2 billion iExo). While the siRNA targets both human and mouse STAT3, the ASO is designed to target human STAT3, and presents with 3 nucleotide mis-matches against the mouse sequence. ASO design included an unmodified (umASO) and modified ASO (mASO, see Methods). Controls included untreated mice and mice treated with exosomes containing non-targeting control siRNA (siCntrl) or ASO (modified Scramble [mASO Scrbl] or umASO STAT3). iExosomes targeting STAT3 using siRNA or mASO significantly reduced Stat3 expression in fibrotic livers upon treatment with both 1 μg/l billion iExo^{siRNA-STAT3} and 5 μg/2 billion iExo^{siRNA-STAT3} or iExo^{mASO-STAT3} (FIGS. 6A and 6B). Superior efficacy was observed at 5 μg/2 billion iExo^{siRNA-STAT3 or} iExo^{mASO-STAT3} compared to siRNA (siRNA-STAT3) or ASO (mASO-STAT3) alone (FIGS. 6A and 6B). umASO did not significantly suppress Stat3 in vivo, possible as a result of diminished stability in this setting, whereas the enhanced stability of the mASO correlated with robust targeting of Stat3 (FIGS. 6A and 6B). Both siRNA and mASO targeting iExosomes showed similar efficacy in suppressing Stat3 expression in fibrotic liver (FIGS. 6A and 6B).

Repetitive exposure to the hepatotoxin CCl₄ induces prominent inflammation and liver damage, which drive a progressive fibrosis and accumulation of activated HSCs or myofibroblasts (Mederacke et al., 2013). Sirius red staining and collagen I staining of liver sections were applied to assess the extracellular matrix (ECM) deposition. The results showed a significant reduction in ECM in mice treated with 5 μg/2 billion iExo^{siRNA-STAT3} or iExo^{mASO-STAT3}, whereas a modest reduction in fibrosis was observed in mice treated with 1 μg/l billion iExo^{siRNA-STAT3} or iExo^{mASO-STAT3} (FIGS. 6C-6H, FIGS. 5D and E). Type I collagen deposition was also inhibited significantly by treatment with 5 μg/2 billion iExo^{siRNA-STAT3} or iExo^{mASO-STAT3} (FIGS. 6E-H). Notably, the expression pattern of α-SMA, a well-established marker of activated HSCs (aHSCs) in the fibrotic livers (FIG. 4B), was significantly inhibited in mice treated with 5 μg/2 billion iExo^{siRNA-STAT3} or iExo^{mASO-STAT3} as compared to mice treated with siRNA-STAT3 or mASO-STAT3 alone (FIG. 6J), while no significant reduction was observed in mice treated with 1 μg/l billion iExo^{siRNA-STAT3} or iExo^{mASO-STAT3} (FIG. 5H).

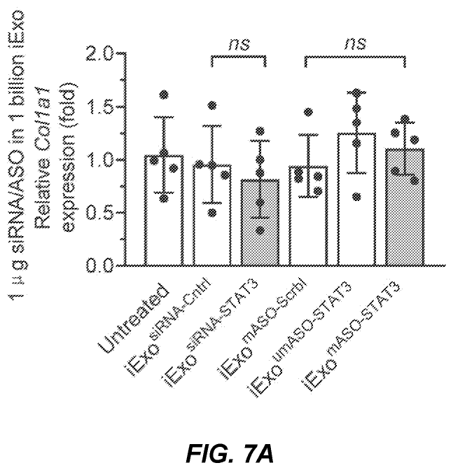

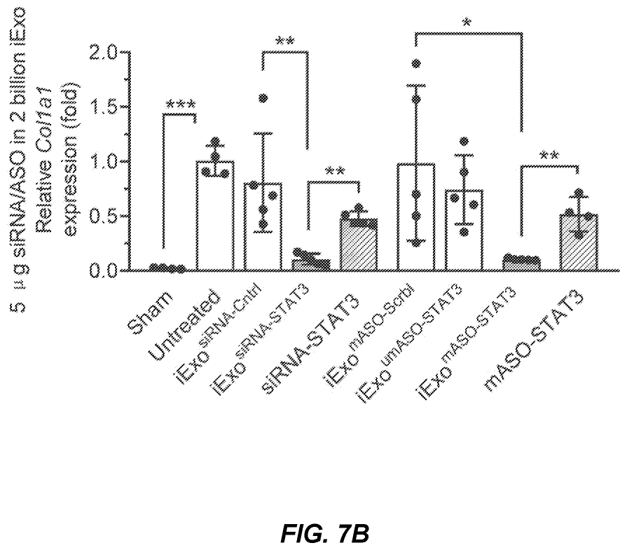

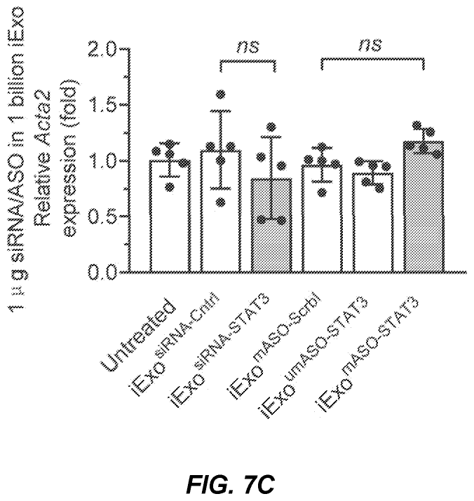

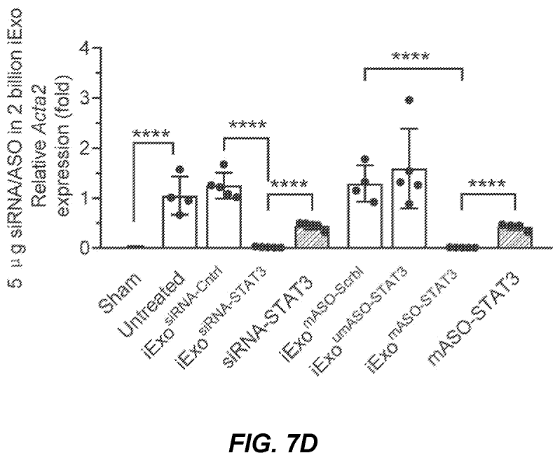

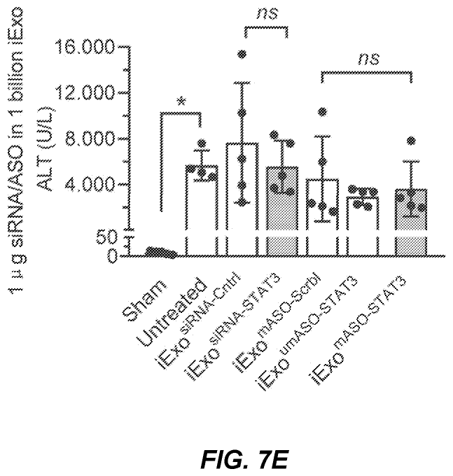

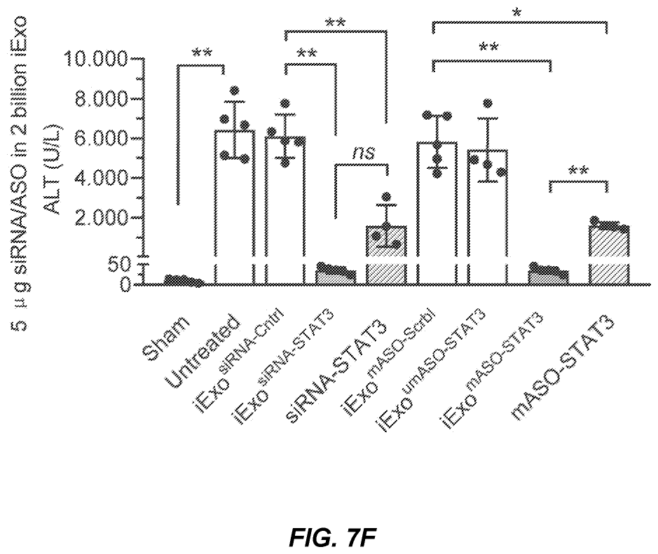

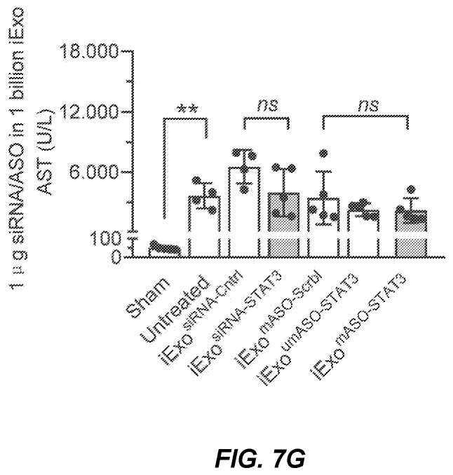

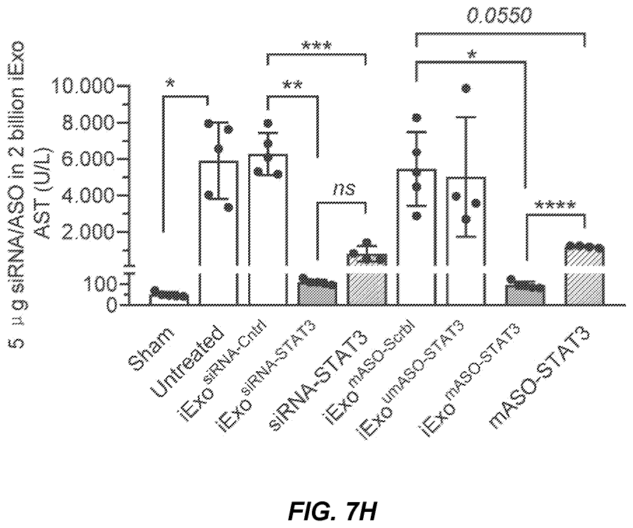

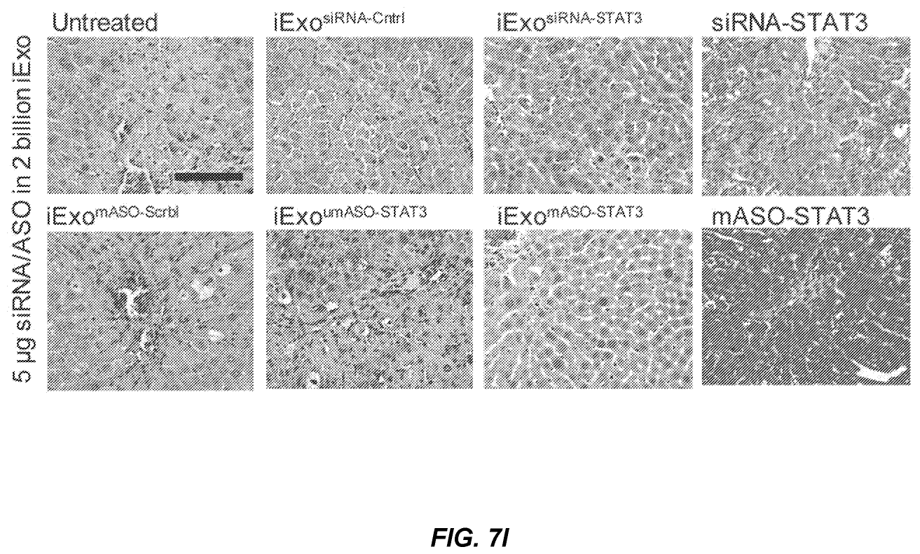

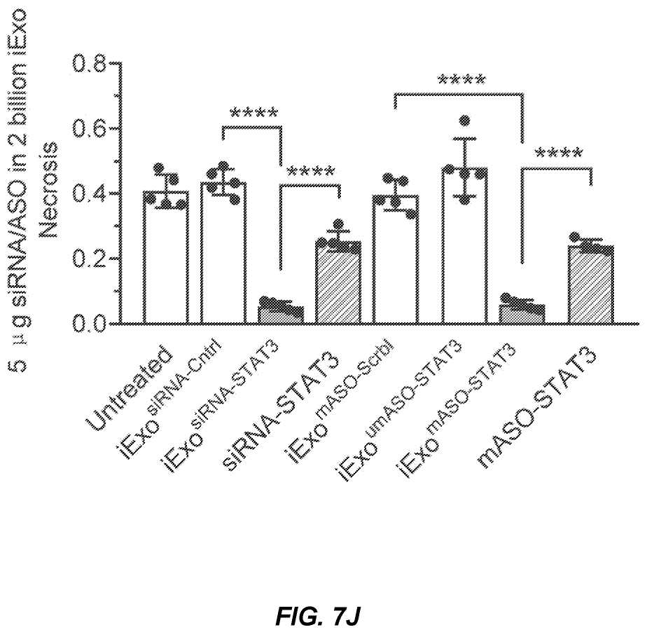

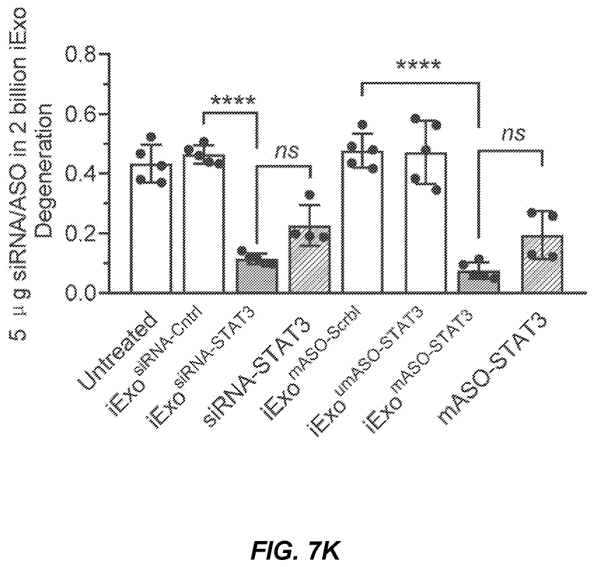

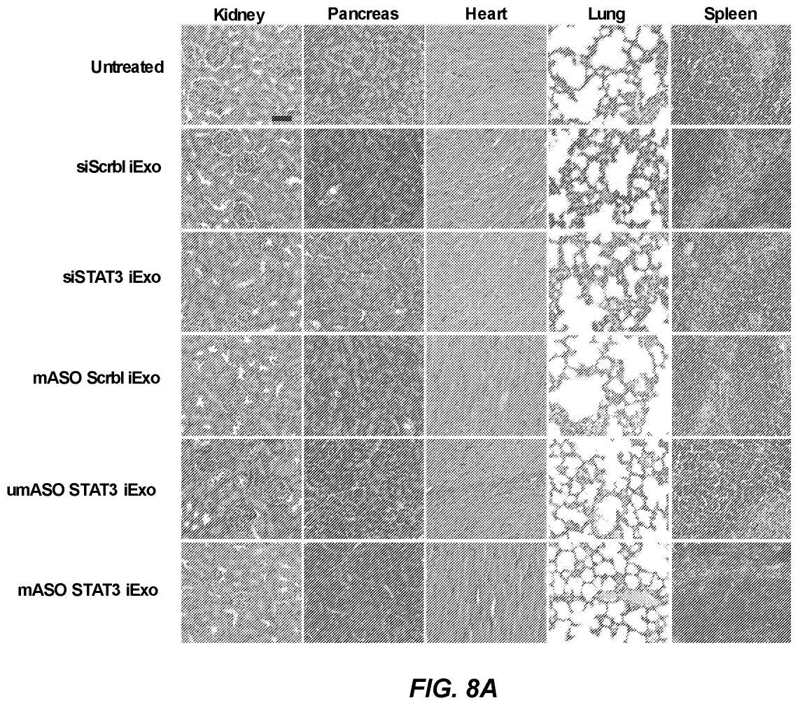

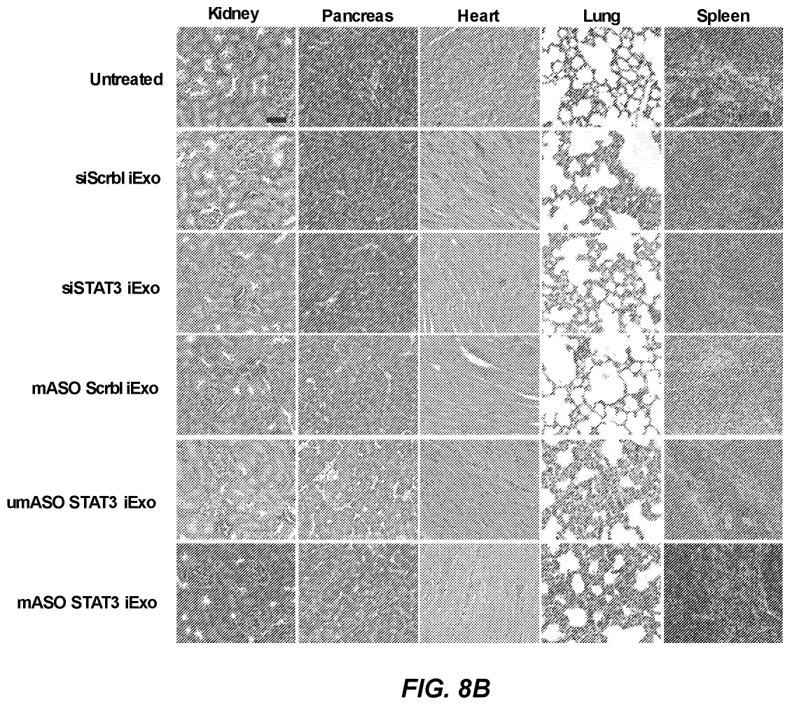

In addition to the downregulation of STAT3 at the transcriptional level, treatment with 5 μg/2 billion iExo^{siRNA-STAT3} or iExo^{mASO-STAT3} resulted in a significant transcriptional suppression of alpha 1 chain of type I collagen (Col1a1) and smooth muscle actin (Acta2), compared to treatment with siRNA-STAT3 or mASO-STAT3 (FIGS. 7A-7D). Liver function was significantly improved in mice treated with 5 μg/2 billion iExo^{siRNA-STAT3} and iExo^{mASO-STAT3} and to some extent with treatment with siRNA-STAT3 or mASO-STAT3 also, as measured by alanine aminotransferase (ALT) and aspartate aminotransferase (AST) levels (FIGS. 7E-7H). Both ALT and AST levels were reduced to levels nearing those of healthy control mice with 5 μg/2 billion iExo^{siRNA-STAT3} and iExo^{mASO-STAT3} treatment (FIGS. 7E-7H). Histopathological evaluation of CCl₄-induced hepatic fibrosis showed hepatocyte degeneration, focal bridging necrosis, and significant structural disruption of the lobule architecture (FIGS. 7I-7K, Untreated group). It was noted that the percentage of hepatocyte necrosis and degeneration was significantly reduced when mice were administered 5 μg/2 billion iExo^{siRNA-STAT3} and iExo^{mASO-STAT3} compared to control treatments, including treatment with siRNA-STAT3 or mASO-STAT3 (FIGS. 7I-7K). Insignificant improvement in liver histopathology was observed in mice when administered 1 μg/l billion iExo^{siRNA-STAT3 or} iExo^{mASO-STAT3} (FIGS. 5I-5K). iExo^{siRNA-STAT3} or iExo^{mASO-STAT3} treatment did not result in observable cytotoxicity to other organs, (FIGS. 8A and 8B).

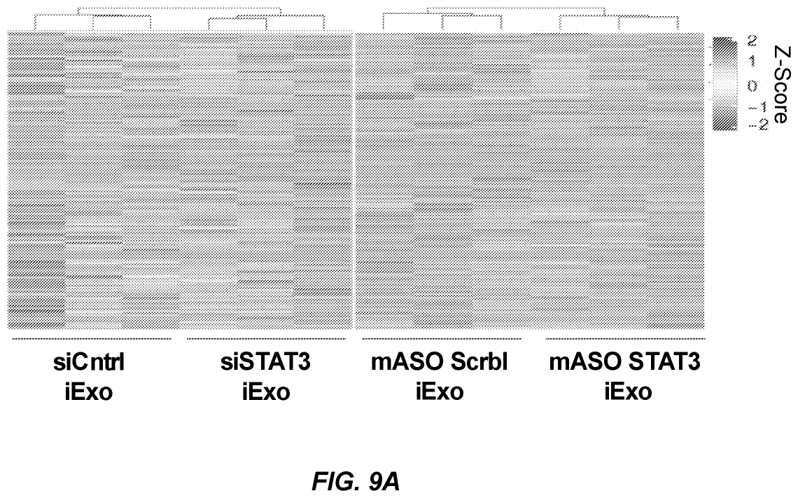

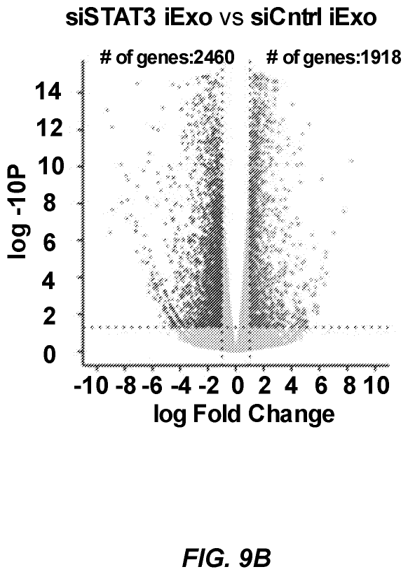

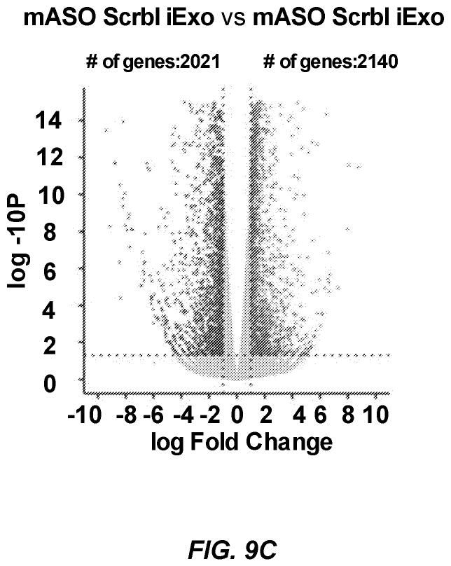

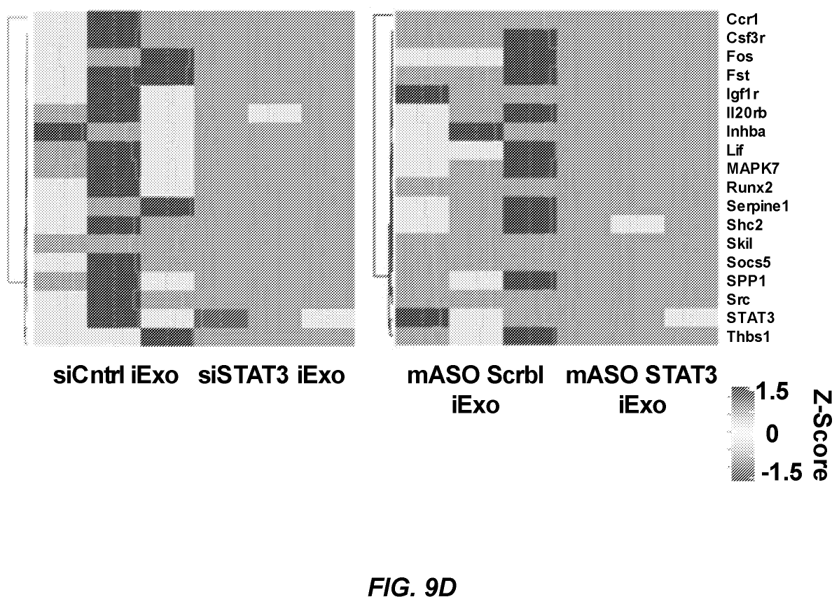

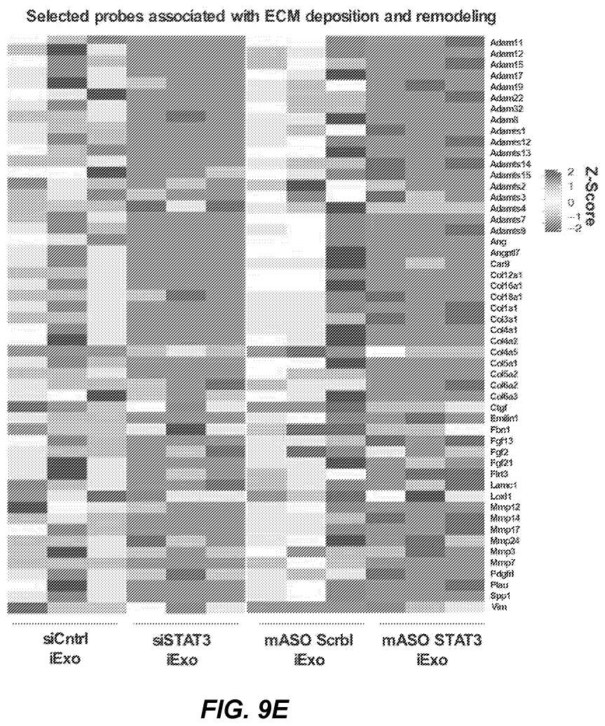

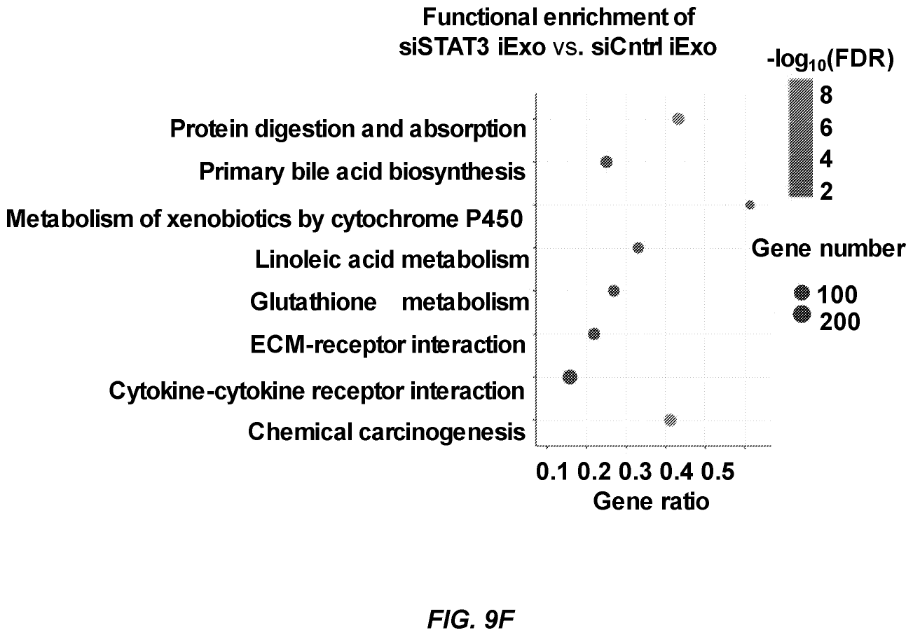

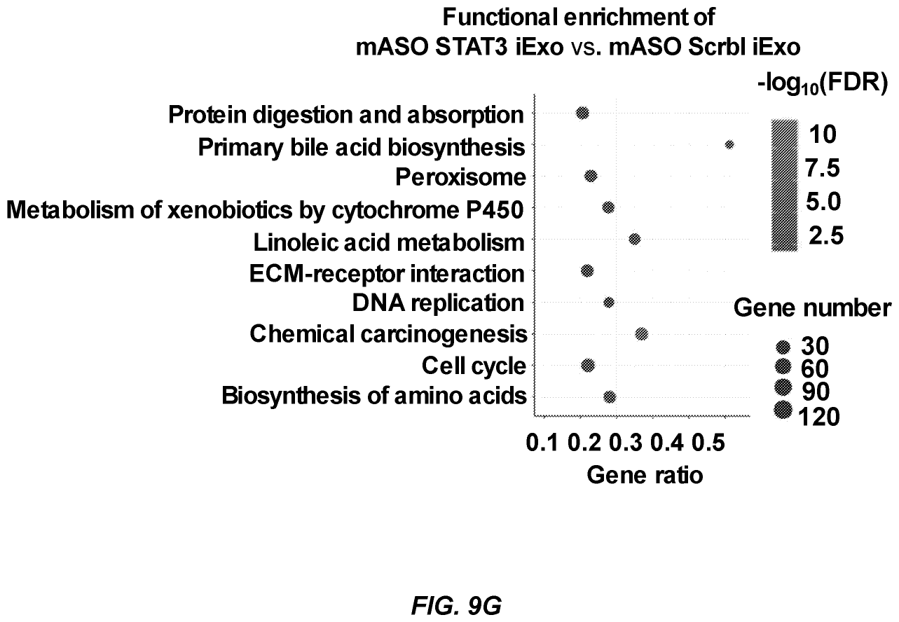

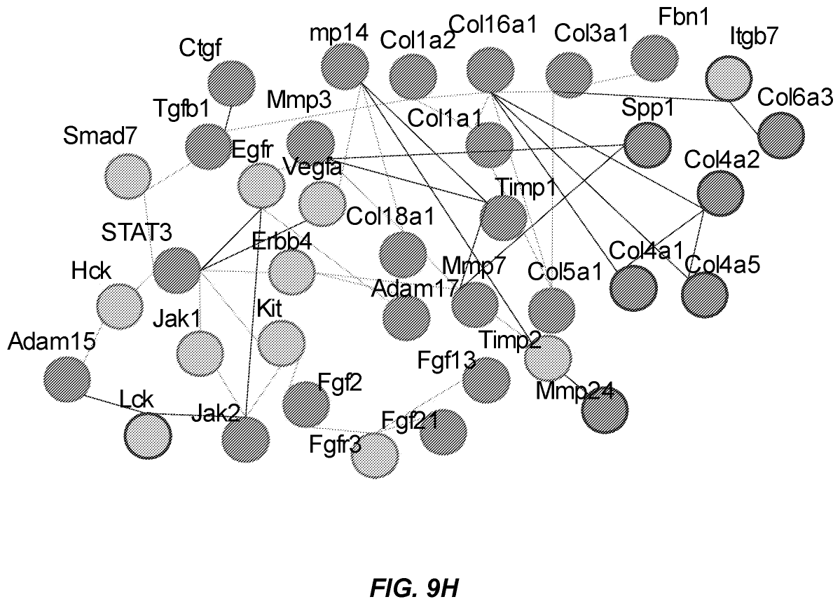

To determine the impact of iExosomes treatment on target cell gene expression, the inventors carried out RNA sequencing of the whole livers from 5 μg/2 billion iExo^{siRNA-STAT3} and iExo^{mASO-STAT3} treated mice. Differentially expressed genes (DEGs) in each experimental group were plotted in a heat map (FIG. 9A), and the significant change in the expression of a given gene was defined with a ratio greater than two-fold increase or decrease and an adjusted p-value <0.05. The heat map and volcano plot indicated that iExo^{siRNA-STAT3} and iExo^{mASO-STAT3} treatment resulted in gene expression changes when compared to their respective controls (FIGS. 9A-9C). Liver transcript analyses from the iExo^{siRNA-STAT3} group showed the increased expression of 1,918 genes, and the decreased expression of 2,460 genes, whereas liver transcript analyses in iExo^{mASO-STAT3} group showed an increase in expression of 2,140 genes and a decrease in expression of 2,021 genes (FIGS. 9B and 9C). A cluster of genes involved in STAT3 signaling were suppressed following iExosomes treatment, including SPP1 and Thbs1 (Arriazu et al., 2017; Breitkopf et al., 2005), which play a vital role in the development of liver fibrosis (FIG. 9D). Commonly differentially deregulated genes related to STAT3 signaling in liver fibrosis were associated with ECM deposition and remodeling (FIG. 9E). iExosomes treatment repressed the expression of canonical fibrosis-associated genes, including Col1a1, Acta2, Col1a2, and Vim (FIG. 9E), suggesting that STAT3 is a key mediator of liver fibrosis. Over-representation analysis demonstrated that the DEGs were mainly enriched in ECM-receptor interaction pathway (FIGS. 9F and 9G) and also indicated that the downregulated genes were enriched for pathways involved in metabolism of xenobiotics by cytochrome P450, protein digestion and absorption, primary bile acid biosynthesis, linoleic acid metabolism, and chemical carcinogenesis (FIGS. 9F and 9G). A similar set of altered downstream pathways was observed for both iExo^{sIRNA-STAT3} and iExo^{mASO-STAT3} treat, emt (FIGS. 9F and 9G), and supports this dataset as a useful tool for further inquiry into STAT3 regulated pathways in liver fibrosis. To further investigate the association between STAT3 signaling and targeted ECM genes in liver fibrosis, an ECM regulatory network associated with STAT3 mRNA and liver fibrosis was constructed based on DEGs. As shown in FIG. 9H, the network generated displayed a connection in 24 ECM-associated genes. This network analysis identified STAT3 as an important node of regelation for ECM deposition for future clinical treatment.

Taken together, these studies support previous reports on the critical role of STAT3 in hepatocytes and stellate cells in promoting liver fibrosis (Wang, Lafdil, Kong et al., 2011). STAT3 deregulation in liver fibrosis is complex, with a protective function in hepatocytes, and a pro-fibrotic in aHSCs/myofibroblasts Chakraborty et al., 2017; Wang, Lafdil, Kong et al., 2011; Wang, Lafdil, Wang et al., 2011). The anti-fibrotic outcome of the iExosomes approach to target STAT3 may reflect a preferential uptake by aHSCs/myofibroblasts (FIG. 5C). This is also in accordance with previous reports using exosomes from adipose-derived mesenchymal stem cells, which were shown to prevent liver fibrosis via exosomal miR-181-5p that suppress STAT3 expression (Qu et al., 2017). Although various inhibitors have shown efficacy in mice, their specificity in targeting STAT3 remains to be validated (Beebe et al., 2018). The disclosed approach offers gene targeting specificity and may be used in combination with additional siRNA targets. Furthermore, the role of exosomes in the therapeutic approaches for liver cancer has also emerged (Lou et al., 2020). Transformed hepatocytes in liver cancer rely on STAT3 expression (Wang, Lafdil, Wang et al., 2011; Jung et al., 2017), and iExosomes targeting STAT3 could also provide benefits in limiting liver cancer progression. Previous studies on pancreatic cancer have set forth the development of clinical grade exosomes with siRNA targeting of oncogenic Kras (Mendt et al., 2018; Kamerkar et al., 2017). This supports the potential of a clinical application for the inhibition of STAT3 using exosomes in liver fibrosis.

Methods

HSCs Isolation and α-SMA Staining

Mouse primary HSCs were isolated from 8-week-old female Balb/c mice according to the methods previously described (Vinas et al., 2003). They were cultured in high-glucose Dulbecco's modified Eagle's medium (DMEM, Gibco) containing 20% FBS (Gemini). The culture-activated primary HSCs (on the day 7) were immunostained with Cy3-α-SMA antibody (Sigma, C6198, 1:200) overnight. Representative images at 200× magnification were taken with Axiovert 200 and Axiocam HRc camera (Zeiss).

Real-Time PCR Analyses

Total RNA was isolated from liver tissue using TRIzol™ (Invitrogen, 15596018) and cDNA was generated via the High-Capacity cDNA Reverse Transcriptase Kit (Life Technology) according to the manufacturers' instructions. Quantitative RT-PCR was performed using SYBR Green PCR Master Mix. Total amount of mRNA of the target genes was normalized to GAPDH expression. The following primer sequences were used: GAPDH Forward 5′-CTGGAGAAACCTGCCAAGTA-3′. Reverse 5′-AAGAGTGGGAGTTGCTGTTG-3′. STAT3 Forward 5′-AGAACCTCCAGGACGACTTTG-3′, Reverse 5′-TCACAATGCTTCTCCGCATCT-3′; Col1a1 Forward 5′-CATGTTCAGCTTTGTGGACCT-3′, Reverse 5′-GCAGCTGACTTCAGGGATGT-3′; Acta2 Forward 5′-GTCCCAGACATCAGGGAGTAA-3′, Reverse 5′-TCGGATACTTCAGCGTCAGGA-3′. Statistical analyses for variance were performed on the ΔCt. The fold change is presented and normalized to the control group, setting the control comparative group to 1.

Purification and Electroporation of Exosomes

Bone marrow-derived MSCs were obtained from the Cell Therapy Laboratory at the University of Texas MD Anderson Cancer Center and cultured in αMEM (Corning) supplemented with 20% FBS, 1% penicillin-streptomycin (Corning), 1% L-glutamine (Corning), and 1% non-essential amino acids (NEAA, Gibco). Passage 4 to 6 MSCs were used for exosome collections. Exosomes were purified by differential centrifugation processes according to our established protocols (Mendt et al., 2018; Kamerkar et al., 2017). Briefly, cells were grown to 70-80% confluency, washed with 1×PBS (Corning), and cultured in serum-free media (αMEM with 1% penicillin-streptomycin, 1% L-glutamine, and 1% NEAA) for 48 hours. Supernatant was collected, centrifuged at 800×g for 5 minutes followed by 2,000×g for 10 minutes, and filtered with a 0.2 μm filter (Thermo Fisher). Filtered supernatant was centrifuged at 100,000×g in a SW 32 Ti rotor (Beckman) for 3 hours at 4° C. The supernatant was aspirated, and the pellet was resuspended in 100 μL of 1×PBS. Exosomes concentration and size were verified by nanoparticle tracking analysis (NTA, NanoSight LM10, Malvern). Aliquots of 10 billion exosomes were stored at −80° C. prior to use. Low-dose mixture contained 1 billion of total exosomes according to NTA and 1 μg of siRNA or antisense oligos (ASO) in 100 μl of PlasmaLyte (Medline, BHL2B2544XH), while high-dose mixture contained 2 billion of total exosomes and 5 μg of siRNA or ASO in 100 μl of PlasmaLyte. 400 μl of the RNAi-exosomes mixture was loaded in the cuvette and then electroporated at 400V, 125 μF and ∞ ohms. The cuvette was immediately transferred to ice. The siSTAT3 sequence: sense strand 5′-GUUGAAUUAUCAGCUUAAA-3′ (SEQ ID NO:1), anti-sense 5′-UUUAAGCUGAUAAUUCAAC-3′ (SEQ ID NO:2) (Sigma-Aldrich). The mASO Scrbl sequence was 5′-mG*mG*mC*mU*mA*C*U*A*C*G*C*mC*mG*mU*mC*mA-3′ (SEQ ID NO:3). The umASO STAT3 sequence was 5′-CTATTTGGATGTCAGC-3′ (SEQ ID NO:4). The mASO STAT3 sequence was 5′-mC*mU*mA*mU*mU*U*G*G*A*U*G*mU*mC*mA*mG*mC-3′ (SEQ ID NO:5). ‘m’ denotes 2′ O-methoxy-ethyl bases, * denotes phosphorothioate bonds. The siCntrl was obtained from Sigma-Aldrich (SIC001, Sigma-Aldrich). The ASOs were synthesized by Integrated DNA Technologies, Inc. The siRNA was designed with equal potential efficiency to target mouse and human STAT3. The ASO was also designed to target mouse and human STAT3, but with potentially lower efficacy against mouse STAT3 due to a 3 nucleotides mismatch with the mouse sequence.

Visualization of exosome biodistribution in vivo.

Mice were treated with CCl₄ to induce liver fibrosis (as detailed below). For the biodistribution of MSC-derived exosomes, 8×10⁹ purified exosomes labeled with XenoLight DiR (1,1′-dioctadecyltetramethyl indotricarbocyanine iodide, Perkin Elmer, catalog 125964) were injected i.p (100 μl) in healthy (sham) and fibrotic Balb/c mice as previously described (Mendt et al., 2018). Diluted DiR (100 μl) was injected as a control. After 6 hours of injection, the mice were euthanized, and various tissues (kidneys, spleen, liver, pancreas and bowel) were harvested and imaged immediately. Briefly, every 5×10⁹ MSC-derived exosomes were labeled with 1 μM DiR, and then incubated for 1 hour at 37° C. and 15 minutes at 4° C. and then washed at 4° C. for 3 hours by ultracentrifugation 40,000 g in 10 ml of 1×PBS (Mendt et al., 2018). The labeled exosomes (8×10⁹) were resuspended in 100 μl of 1×PBS. For the control group, 1 μl of DiR was diluted in 11 ml 1×PBS by ultracentrifugation for 3 hours according to the same procedure as above. Control samples (only DiR) were resuspended in 100 μl 1×PBS. The fluorescent intensity of variant organs was imaged and quantified by using the IVIS 200 small animal imaging system (PerkinElmer) with the Em filter at 780 nm and the Ex filter at 710 nm.

Visualization of Labeled siSTAT3 and mASO STAT3 Localization in the Liver Tissue

Mice were treated with CCl₄ to induce liver fibrosis (as detailed below). The MSC-derived exosomes were electroporated with AF647 tagged siRNA and mASO prior to the injection. These AF647 labeled iExosomes and AF647 tagged naked siRNA or mASO were then injected i.p. into WT Balb/c and fibrotic mice. Sectioned liver specimen were stained with DAPI and then mounted. Images were obtained by confocal laser scanning microscope (Zeiss LSM800) and then quantified by counting the number of nuclei of AF647 positive cells and divided by the total number of nuclei. Three random visual fields were captured per organ (200×).

Mice

Liver fibrosis was induced in Balb/c mice (8-week old female purchased from the Jackson laboratory). Liver injury was induced with i.p. injections of CCl₄ (Sigma, 56-23-5) at a dosage of 10% in 100 μl olive oil twice a week for 37 days. Control mice were administered with olive oil devoid of CC14 (FIG. 5D). 9 days later, the mice were randomly assigned into 13 groups. Mice were also administered 1 μg siRNA/ASO of 1 billion engineered exosomes or 5 μg siRNA/ASO of 2 billion engineered exosomes i.p. in 100 μl volume of PlasmaLyte (Medline) or siRNA-STAT3/mASO-STAT3 alone every other day. The mice were euthanized within 24 h after the last iExosomes injection. All protocols and procedures were approved by the Institute for Animal Care and Use Committee at MDACC.

Sirius Red Staining and Quantification

Livers were fixed in 10% neutral buffered formalin and embedded in paraffin. 5 μm in thickness paraffin sections were used for Sirius red staining. After being rinsed for three times and stained with Weigert's haematoxylin for 8 min, the slides were counterstained by picrosirius red for 1 hour. To quantify liver fibrosis, three independent Sirius red-stained sections were analyzed from each mouse using a counting grid. The percent area of liver fibrosis was calculated as previously described (Whittaker et al., 1994).

Immunohistochemistry

Tissues were fixed in 10% formalin overnight, dehydrated, and embedded in paraffin. 5 μm sections were then processed for analyses. Heat-mediated antigen retrieval in 1 mM EDTA-TE (pH 9.0) for 1 hour was performed. Sections for Collagen I (Southern Biotech, 1310-01, 1:200) staining were blocked with 4% CWFS gelatin (Aurion) in TBS, 1 hour prior to overnight incubation with the primary antibodies. After incubated the biotinylated anti-goat (Vector Laboratories, BA9500, 1: 400), the sections were reacted with ABC for half an hour and then developed by DAB according to the manufacturer's protocol.

Liver Function Evaluation

Mice blood was collected from the retro-orbital plexus. Serum was then immediately isolated by centrifugation 6,000 rpm at 4° C. for 10 min and stored at −80° C. until use. The measurements of ALT and AST contents of the serum were performed by the department of Veterinary Pathology at MDACC.

Haematoxylin and Eosin Staining

Liver tissue samples were fixed in 10% buffered formalin and embedded in paraffin. Tissue sections at a thickness of 5 μm were stained with haematoxylin and eosin (H&E). Five distinct 200× visual fields were randomly selected for each slide and the number of necrotic and degenerated hepatocytes was manually counted using the count tool of Adobe Photoshop 7.0. Hepatocytes were defined as necrotic according to condensation and dark staining of the cytoplasm and absence of nucleus (Krishna et al., 2017). Hepatocytes degeneration was determined by cell swelling and enlargement found particularly as previously reported (Lackner et al., 2008).

RNA Sequencing

Total RNA was extracted from livers using TRIzol™ (Invitrogen, 15596018) and purified according to manufacturer instructions. RNA integrity was determined using RNA 6000 Nano Assay by the MDACC Sequencing and ncRNA Program core. Liver RNA sequencing was performed using Illumina TrueSeq stranded mRNAseq MDACC Sequencing and ncRNA Program core. Genome mapping was performed using TopHat software (v2.0.9; available on the World Wide Web at ccb.jhu.edu/software/tophat/index.shtml). The inventors used Cufflinks algorithm for identification of transcripts from RNA-Seq data and determined differentially expressed genes using DESeq2 (available on the World Wide Web at bioconductor.org/packages/release/bioc/html/DESeq2.html) for gene expression profiling. False-discovery rate (FDR) was performed to determine the significance threshold of the p-value for multiple tests. The significant expressed genes were determined by FDRs less than 0.05. Gene Set Enrichment Analysis (GSEA) and gene annotation were conducted by WebGestalt 2019 (available on the World Wide Web at webgestalt.org/) (He et al., 2019). The STAT-ECM genes interaction regulatory network was constructed using NetworkAnalyst 3.0 (available on the World Wide Web at networkanalyst.ca/) (Zhou et al., 2019).

Statistical Analyses

Statistical analyses used are detailed in the Brief Description of the Drawings. Data are expressed as mean±standard error of the mean. p<0.05 was considered statistically significant. One-way ANOVA or unpaired two-tailed Student's t-test with Welch's correction were used to establish statistical significance using GraphPad Prism (GraphPad Software). Significance of statistical tests is reported in graphs as follows: ****(p<0.0001), ***(p<0.001), **(p<0.01), *(p<0.05), n.s. (p>0.05).

### Example 5 —Col1a1 Knockout in Hepatic Stellate Cells

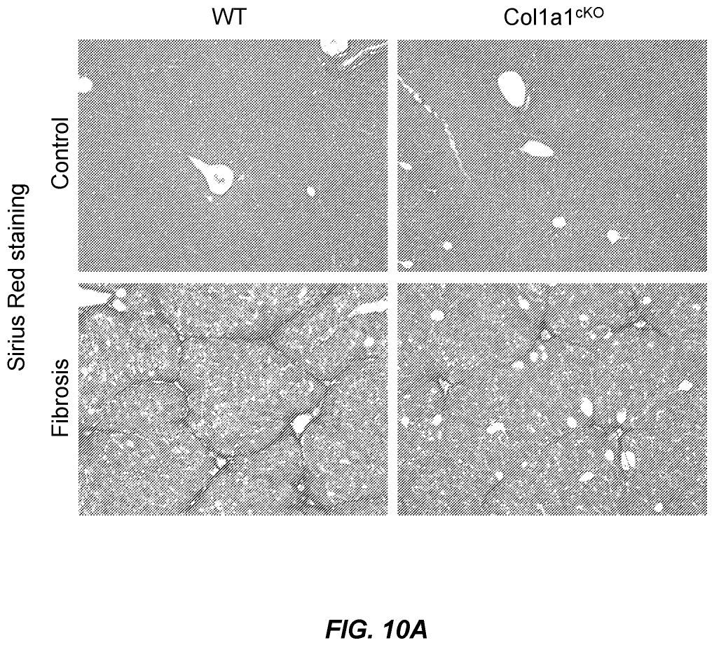

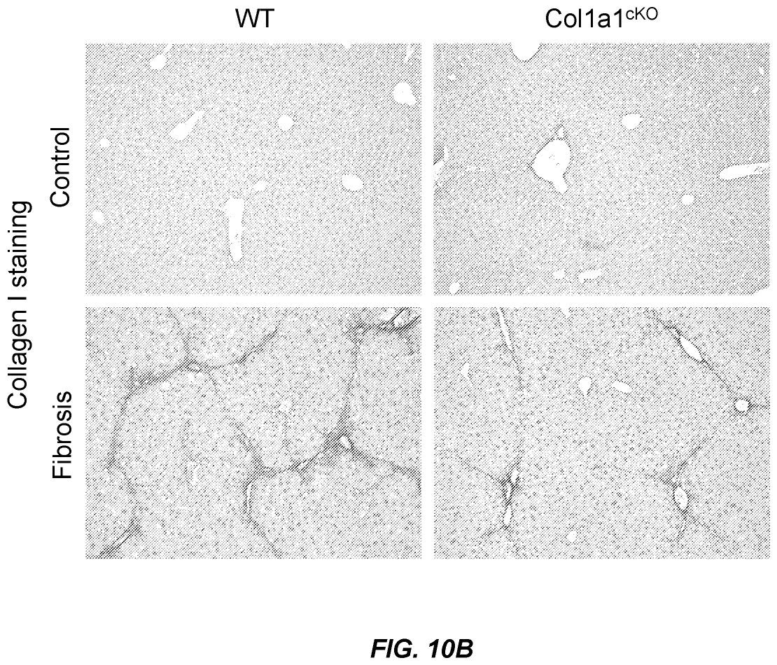

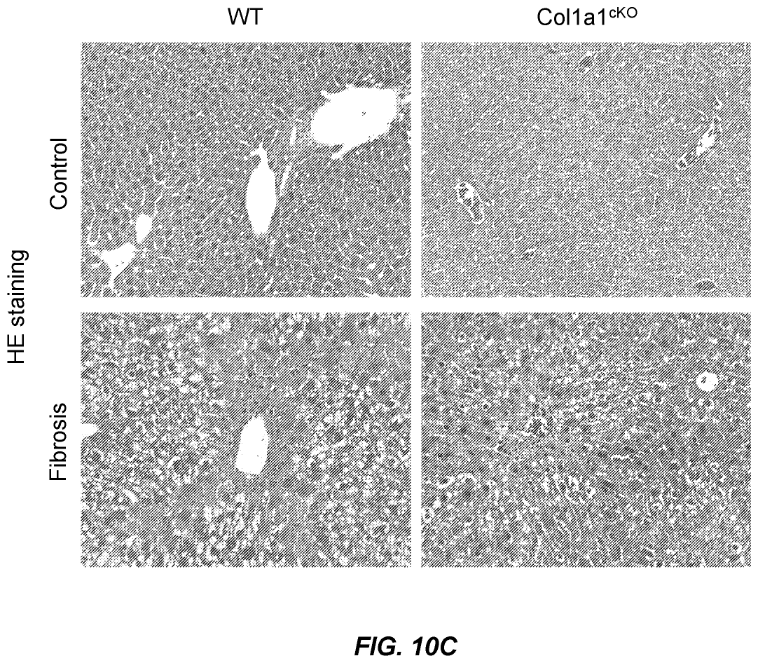

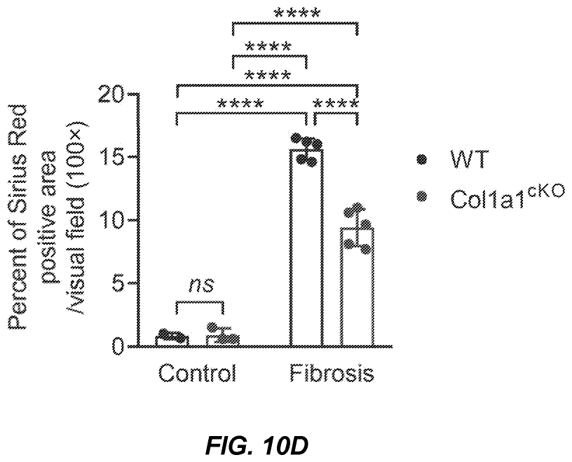

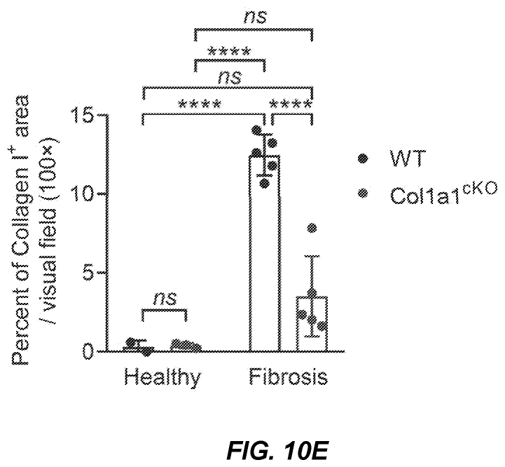

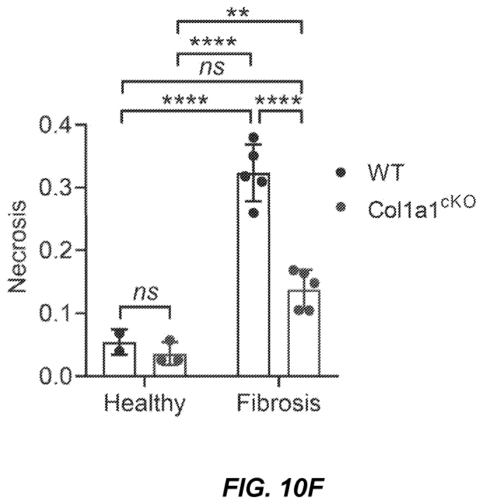

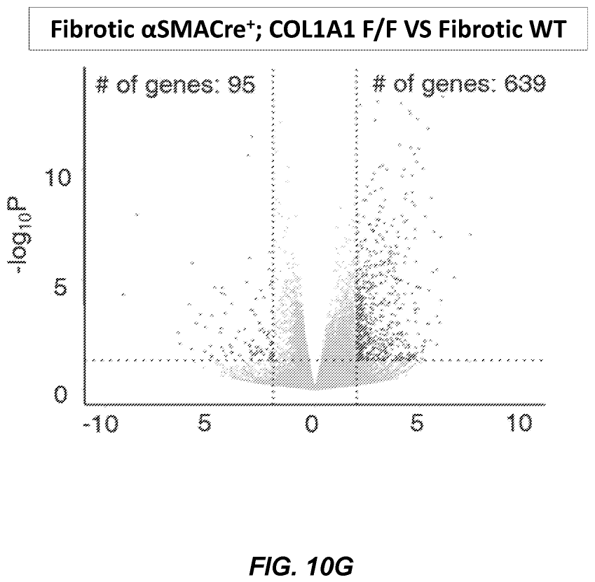

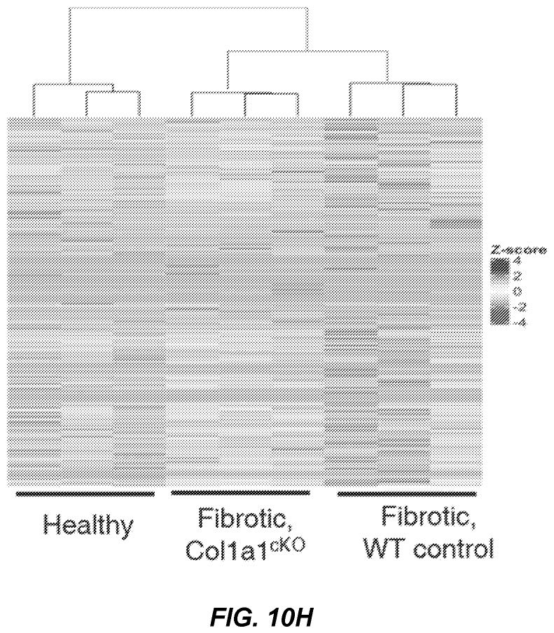

Mice were generated harboring a knockout mutation of the Col1a1 gene in activated hepatic stellate cells or αSMA⁺ myofibroblasts (Col1a1^{cKO}). Liver fibrosis was induced in one group of mice with i.p. injections of CCl₄, while the control group did not receive CCl4. and both fibrosis and control (“healthy”) groups were sacrificed and analyzed. A significant reduction in Collagen I and liver fibrosis was observed in Col1a1^{cKO} mice compared with wild type (WT) (FIGS. 10A-F). Many of the global expression patterns associated with liver fibrosis were significantly improved in the Col1a1^{cKO} mice with liver fibrosis compared with WT (FIGS. 10G and 10H).

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. For example, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Arriazu E, Ge X, Leung™, Magdaleno F, Lopategi A, Lu Y, et al. Signalling via the osteopontin and high mobility group box-1 axis drives the fibrogenic response to liver injury. Gut. 2017; 66(6):1123-37.
Bartneck M, Warzecha K T, and Tacke F. Therapeutic targeting of liver inflammation and fibrosis by nanomedicine. Hepatobiliary Surg Nutr. 2014; 3(6):364-76.
Bataller R, and Brenner D A. Liver fibrosis. J Clin Invest. 2005; 115(2):209-18.
Beebe J D, Liu J Y, and Zhang J T. Two decades of research in discovery of anticancer drugs targeting STAT3, how close are we? Pharmacol Ther. 2018; 191:74-91.
Breitkopf K, Sawitza I, Westhoff J H, Wickert L, Dooley S, and Gressner A M. Thrombospondin 1 acts as a strong promoter of transforming growth factor beta effects via two distinct mechanisms in hepatic stellate cells. Gut. 2005; 54(5):673-81.
Chakraborty D, Sumova B, Mallano T, Chen C W, Distler A, Bergmann C, et al. Activation of STAT3 integrates common profibrotic pathways to promote fibroblast activation and tissue fibrosis. Nat Commun. 2017; 8(1):1130.
Choi S, Jung H J, Kim M W, Kang J H, Shin D, Jang Y S, et al. A novel STAT3 inhibitor, STX-0119, attenuates liver fibrosis by inactivating hepatic stellate cells in mice. Biochem Biophys Res Commun. 2019; 513(1):49-55.
Gong X H, Chen C, Hou P, Zhu S C, Wu C Q, Song C L, et al. Overexpression of miR-126 inhibits the activation and migration of HSCs through targeting CRK. Cell Physiol Biochem. 2014; 33(1):97-106.
He W, Fu L, Yan Q, Zhou Q, Yuan K, Chen L, et al. Gene set enrichment analysis and meta-analysis identified 12 key genes regulating and controlling the prognosis of lung adenocarcinoma. Oncol Lett. 2019; 17(6):5608-18.
Hernandez-Gea V, and Friedman S L. Pathogenesis of liver fibrosis. Annu Rev Pathol. 2011; 6:425-56.
Jung K H, Yoo W, Stevenson H L, Deshpande D, Shen H, Gagea M, et al. Multifunctional Effects of a Small-Molecule STAT3 Inhibitor on NASH and Hepatocellular Carcinoma in Mice. Clin Cancer Res. 2017; 23(18):5537-46.
Kamerkar S, LeBleu V S, Sugimoto H, Yang S, Ruivo C F, Melo S A, et al. Exosomes facilitate therapeutic targeting of oncogenic KRAS in pancreatic cancer. Nature. 2017; 546(7659):498-503.
Krishna M. Patterns of necrosis in liver disease. Clin Liver Dis (Hoboken). 2017; 10(2):53-6.
Lackner C, Gogg-Kamerer M, Zatloukal K, Stumptner C, Brunt E M, and Denk H. Ballooned hepatocytes in steatohepatitis: the value of keratin immunohistochemistry for diagnosis. J Hepatol. 2008; 48(5):821-8.
Lou G, Chen L, Xia C, Wang W, Qi J, Li A, et al. MiR-199α-modified exosomes from adipose tissue-derived mesenchymal stem cells improve hepatocellular carcinoma chemosensitivity through mTOR pathway. J Exp Clin Cancer Res. 2020; 39(1):4.
Lu Y, Lv F, Kong M, Chen X, Duan Y, Chen X, et al. A cAbl-MRTF-A Feedback Loop Contributes to Hepatic Stellate Cell Activation. Front Cell Dev Biol. 2019; 7:243.
Mederacke I, Hsu C C, Troeger J S, Huebener P, Mu X, Dapito D H, et al. Fate tracing reveals hepatic stellate cells as dominant contributors to liver fibrosis independent of its aetiology. Nat Commun. 2013; 4:2823.
Mendt M, Kamerkar S, Sugimoto H, McAndrews K M, Wu C C, Gagea M, et al. Generation and testing of clinical-grade exosomes for pancreatic cancer. JCI Insight. 2018; 3(8).
Meng X M, Nikolic-Paterson D J, and Lan H Y. TGF-beta: the master regulator of fibrosis. Nat Rev Nephrol. 2016; 12(6):325-38.
Pechkovsky D V, Prele C M, Wong J, Hogaboam C M, McAnulty R J, Laurent G J, et al. STAT3-mediated signaling dysregulates lung fibroblast-myofibroblast activation and differentiation in UIP/IPF. Am J Pathol. 2012; 180(4):1398-412.
Qu Y, Zhang Q, Cai X, Li F, Ma Z, Xu M, et al. Exosomes derived from miR-181-5p-modified adipose-derived mesenchymal stem cells prevent liver fibrosis via autophagy activation. J Cell Mol Med. 2017; 21(10):2491-502.
Su T H, Shiau C W, Jao P, Liu C H, Liu C J, Tai W T, et al. Sorafenib and its derivative SC-1 exhibit antifibrotic effects through signal transducer and activator of transcription 3 inhibition. Proc Natl Acad Sci USA. 2015; 112(23):7243-8.
Vinas O, Bataller R, Sancho-Bru P, Gines P, Berenguer C, Enrich C, et al. Human hepatic stellate cells show features of antigen-presenting cells and stimulate lymphocyte proliferation. Hepatology. 2003; 38(4): 919-29.
Wang H, Lafdil F, Kong X, and Gao B. Signal transducer and activator of transcription 3 in liver diseases: a novel therapeutic target. Int J Biol Sci. 2011; 7(5):536-50.
Wang H, Lafdil F, Wang L, Park O, Yin S, Niu J, et al. Hepatoprotective versus oncogenic functions of STAT3 in liver tumorigenesis. Am J Pathol. 2011; 179(2):714-24.
Whittaker P, Kloner R A, Boughner D R, and Pickering J G. Quantitative assessment of myocardial collagen with picrosirius red staining and circularly polarized light. Basic Res Cardiol. 1994; 89(5):397-410.
Xiang D M, Sun W, Ning B F, Zhou T F, Li X F, Zhong W, et al. The HLF/IL-6/STAT3 feedforward circuit drives hepatic stellate cell activation to promote liver fibrosis. Gut. 2018; 67(9):1704-15.
Zeybel M, Luli S, Sabater L, Hardy T, Oakley F, Leslie J, et al. A Proof-of-Concept for Epigenetic Therapy of Tissue Fibrosis: Inhibition of Liver Fibrosis Progression by 3-Deazaneplanocin A. Mol Ther. 2017; 25(1):218-31.
Zhai X, Wang W, Dou D, Ma Y, Gang D, Jiang Z, et al. A novel technique to prepare a single cell suspension of isolated quiescent human hepatic stellate cells. Sci Rep. 2019; 9(1):12757.
Zhou G, Soufan O, Ewald J, Hancock R E W, Basu N, and Xia J. Network Analyst 3.0: a visual analytics platform for comprehensive gene expression profiling and meta-analysis. Nucleic Acids Res. 2019; 47(W1):W234-W41.

What is claimed is:

1. A composition comprising a lipid-based nanoparticle that contains an inhibitory RNA that hybridizes to a STAT3 polynucleotide.

2. The composition of claim 1, wherein the lipid-based nanoparticle comprises CD47 on its surface.

3. The composition of claim 1 or 2, wherein the lipid-based nanoparticle comprises a growth factor on its surface.

4. The composition of any of claims 1-3, wherein the lipid-based nanoparticle is a liposome or an exosome.

5. The composition of any of claims 1-4, wherein the inhibitory RNA is a siRNA, shRNA, antisense oligonucleotide, miRNA, or pre-miRNA.

6. The composition of claim 5, wherein the inhibitory RNA is an antisense oligonucleotide and wherein the antisense oligonucleotide is modified.

7. The composition of any of claims 1-6, wherein the inhibitory RNA knocks down the expression of STAT3 protein.

8. The composition of any of claims 1-7, wherein the inhibitory RNA has a size between 18 and 30 nucleotides.

9. The composition of any of claims 1-8, wherein the inhibitory RNA comprises SEQ ID NO:1.

10. The composition of any of claims 1-8, wherein the inhibitory RNA comprises SEQ ID NO:2.

11. The composition of any of claims 1-8, wherein the inhibitory RNA comprises SEQ ID NO:3.

12. The composition of any of claims 1-8, wherein the inhibitory RNA comprises SEQ ID NO:4.

13. The composition of any of claims 1-8, wherein the inhibitory RNA comprises SEQ ID NO:5.

14. A pharmaceutical composition comprising a composition of any one of claim 1-13 and an excipient.

15. The pharmaceutical composition of claim 14, wherein the composition is formulated for parenteral administration.

16. The pharmaceutical composition of claim 15, wherein the composition is formulated for intravenous, intramuscular, sub-cutaneous, or intraperitoneal injection.

17. The pharmaceutical composition of any of claims 14-16, further comprising an antimicrobial agent.

18. The pharmaceutical composition of claim 17, wherein the antimicrobial agent is benzalkonium chloride, benzethonium chloride, benzyl alcohol, bronopol, centrimide, cetylpyridinium chloride, chlorhexidine, chlorobutanol, chlorocresol, chloroxylenol, cresol, ethyl alcohol, glycerin, exetidine, imidurea, phenol, phenoxyethanol, phenylethl alcohol, phenlymercuric nitrate, propylene glycol, or thimerosal.

19. A method of treating fibrosis or a condition associated with fibrosis in a patient in need thereof comprising administering the pharmaceutical composition of any one of claims 14-18 to the patient.

20. The method of claim 19, wherein administering the pharmaceutical composition results in delivery of the inhibitory RNA to a cell in the patient.

21. The method of claim 19 or 20, wherein the fibrosis is liver fibrosis, lung fibrosis, pulmonary fibrosis, cystic fibrosis, idiopathic pulmonary fibrosis (IPF), or radiation-induced lung injury.

22. The method of claim 21, wherein the fibrosis is liver fibrosis.

23. The method of any of claims 19-21, wherein the pharmaceutical composition is administered via systemic administration.

24. The method of claim 23, wherein the systemic administration is intravenous administration.

25. The method of any of claims 19-24, further comprising administering at least a second therapy to the patient.

26. The method of any of claims 19-25, wherein the patient is a human.

27. The method of claim 26, wherein the lipid-based nanoparticle is an exosome, wherein the exosome is autologous to the patient.

28. The method of any of claims 19-27, wherein administering the pharmaceutical composition reduces expression of Colla1 in hepatic cells of the patient.

29. The method of any of claims 19-27, wherein administering the pharmaceutical composition reduces expression of Acta2 in hepatic cells of the patient.

30. The method of any of claims 19-27, wherein administering the pharmaceutical composition reduces expression of Colla2 in hepatic cells of the patient.

31. The method of any of claims 19-27, wherein administering the pharmaceutical composition reduces expression of Vim in hepatic cells of the patient.

32. The method of any of claims 19-31, wherein the hepatic cells are hepatic stellate cells.

33. The method of any of claims 19-31, wherein the hepatic stellate cells are activated hepatic stellate cells.

34. A composition comprising a lipid-based nanoparticle that contains an inhibitory RNA that hybridizes to a STAT3 polynucleotide.

35. The composition of claim 34, wherein the lipid-based nanoparticle comprises CD47 on its surface.

36. The composition of claim 34, wherein the lipid-based nanoparticle comprises a growth factor on its surface.

37. The composition of claim 34, wherein the lipid-based nanoparticle is a liposome or an exosome.

38. The composition of claim 34, wherein the inhibitory RNA is a siRNA, shRNA, antisense oligonucleotide, miRNA, or pre-miRNA.

39. The composition of claim 38, wherein the inhibitory RNA is an antisense oligonucleotide and wherein the antisense oligonucleotide is modified.

40. The composition of claim 34, wherein the inhibitory RNA knocks down the expression of STATS protein.

41. The composition of claim 34, wherein the inhibitory RNA has a size between 18 and 30 nucleotides.

42. The composition of claim 34, wherein the inhibitory RNA comprises SEQ ID NO:1.

43. The composition of claim 34, wherein the inhibitory RNA comprises SEQ ID NO:2.

44. The composition of claim 34, wherein the inhibitory RNA comprises SEQ ID NO:3.

45. The composition of claim 34, wherein the inhibitory RNA comprises SEQ ID NO:4.

46. The composition of claim 34, wherein the inhibitory RNA comprises SEQ ID NO:5.

47. A pharmaceutical composition comprising a composition of any one of claims 34-46 and an excipient.

48. The pharmaceutical composition of claim 47, wherein the composition is formulated for parenteral administration.

49. The pharmaceutical composition of claim 48, wherein the composition is formulated for intravenous, intramuscular, sub-cutaneous, or intraperitoneal injection.

50. The pharmaceutical composition of claim 48, further comprising an antimicrobial agent.

51. The pharmaceutical composition of claim 50, wherein the antimicrobial agent is benzalkonium chloride, benzethonium chloride, benzyl alcohol, bronopol, centrimide, cetylpyridinium chloride, chlorhexidine, chlorobutanol, chlorocresol, chloroxylenol, cresol, ethyl alcohol, glycerin, exetidine, imidurea, phenol, phenoxyethanol, phenylethl alcohol, phenlymercuric nitrate, propylene glycol, or thimerosal.

52. A method of treating fibrosis or a condition associated with fibrosis in a patient in need thereof comprising administering the pharmaceutical composition of any one of claims 47-51 to the patient.

53. The method of claim 52, wherein administering the pharmaceutical composition results in delivery of the inhibitory RNA to a cell in the patient.

54. The method of claim 52, wherein the fibrosis is liver fibrosis, lung fibrosis, pulmonary fibrosis, cystic fibrosis, idiopathic pulmonary fibrosis (IPF), or radiation-induced lung injury.

55. The method of claim 54, wherein the fibrosis is liver fibrosis.

56. The method of claim 52, wherein the pharmaceutical composition is administered via systemic administration.

57. The method of claim 56, wherein the systemic administration is intravenous administration.

58. The method of claim 52, further comprising administering at least a second therapy to the patient.

59. The method of claim 52, wherein the patient is a human.

60. The method of claim 59, wherein the lipid-based nanoparticle is an exosome, wherein the exosome is autologous to the patient.

61. The method of claim 60, wherein the exosome is obtained from a body fluid sample obtained from the patient.

62. The method of claim 61, wherein the body fluid sample is blood, lymph, saliva, urine, cerebrospinal fluid, bone marrow aspirates, eye exudate/tears, or serum.

63. The method of claim 60, wherein the exosome is obtained from a mesenchymal cell.

64. The method of claim 52, wherein the composition is administered more than once.

65. The method of claim 52, wherein administering the pharmaceutical composition reduces expression of Colla1 in hepatic cells of the patient.

66. The method of claim 52, wherein administering the pharmaceutical composition reduces expression of Acta2 in hepatic cells of the patient.

67. The method of claim 52, wherein administering the pharmaceutical composition reduces expression of Colla2 in hepatic cells of the patient.

68. The method of claim 52, wherein administering the pharmaceutical composition reduces expression of Vim in hepatic cells of the patient.

69. The method of claim 52, wherein the hepatic cells are hepatic stellate cells.

70. The method of claim 52, wherein the hepatic stellate cells are activated hepatic stellate cells.

71. A method of preparing a therapeutic composition comprising introducing an inhibitory RNA that hybridizes to a STAT3 polynucleotide into a lipid-based nanoparticle.

72. The method of claim 71, wherein the lipid-based nanoparticle comprises CD47 on its surface.

73. The method of claim 71 or 72, wherein the lipid-based nanoparticle comprises a growth factor on its surface.

74. The method of any of claims 71-73, wherein the lipid-based nanoparticle is a liposome or an exosome.

75. The method of any of claims 71-74, wherein the inhibitory RNA is a siRNA, shRNA, antisense oligonucleotide, miRNA, or pre-miRNA.

76. The method of claim 75, wherein the inhibitory RNA is an antisense oligonucleotide and wherein the antisense oligonucleotide is modified.

77. The method of any of claims 71-76, wherein the inhibitory RNA knocks down the expression of STAT3 protein.

78. The method of any of claims 71-77, wherein the inhibitory RNA has a size between 18 and 30 nucleotides.

79. The method of any of claims 71-78, wherein the inhibitory RNA comprises SEQ ID NO:1.

80. The method of any of claims 71-78, wherein the inhibitory RNA comprises SEQ ID NO:2.

81. The method of any of claims 71-78, wherein the inhibitory RNA comprises SEQ ID NO:3.

82. The method of any of claims 71-78, wherein the inhibitory RNA comprises SEQ ID NO:4.

83. The method of any of claims 71-78, wherein the inhibitory RNA comprises SEQ ID NO:5.
